(19) United States
(12) Patent Application Publication
(10) Pub. No.: US 2026/0354719 A1
(43) Pub. Date: Nov. 12, 2026

(54) EXAMINING SYSTEM, IMAGE PROCESSING SYSTEM, NON-TRANSITORY COMPUTER-READABLE STORAGE MEDIUM, WEARABLE DEVICE, AND EXAMINING METHOD

(71) Applicant: Panasonic Intellectual Property Management Co., Ltd., Osaka (JP)

(72) Inventor: Motoki YAKO, Osaka (JP)

(73) Assignee: Panasonic Intellectual Property Management Co., Ltd., Osaka (JP)

(21) Appl. No.: 19/658,623

(22) Filed: Apr. 26, 2026

(30) Foreign Application Priority Data
May 7, 2025 (JP) 2025-077486

(51) Int. Cl. A61B 5/1455; A61B 3/00; A61B 3/12; A61B 3/15; A61B 5/00; A61B 5/145

(52) U.S. Cl. CPC A61B 5/14555; A61B 3/0025; A61B 3/12; A61B 3/156; A61B 5/14532; A61B 5/6803

(57) ABSTRACT

An examining system examines biometric information of a subject. The system includes an imaging unit, an image processing circuit, and a determination processing circuit. The imaging unit includes an image sensor and simultaneously captures, by the image sensor, light components in more than three wavelength ranges contained in light reflected from a fundus of the subject to generate image information used to determine the biometric information of the subject. The image processing circuit generates an image of the fundus for each wavelength range from the image information. The determination processing circuit determines the biometric information of the subject based on the image of the fundus for each wavelength range.

### CROSS REFERENCE TO RELATED APPLICATION

This application claims benefit of priority to Japanese Patent Application 2025-077486, filed May 7, 2025, the entire content of which is incorporated herein by reference.

### BACKGROUND

### Technical Field

The present disclosure relates to an examining system, an image processing system, a non-transitory computer-readable storage medium, a wearable device, and an examining method.

### Background Art

JP2021-36938A discloses a measurement apparatus for measuring a subject's blood glucose level. The measurement apparatus of JP2021-36938A includes a first light source that emits a first measurement light, a second light source that emits a second measurement light, a scanning mirror, and a control unit. The first light source is a wavelength-swept laser. The first measurement light is laser light whose wavelength changes over time between 900 nm and 1200 nm. The second light source is a semiconductor laser. The second measurement light is infrared laser light with a wavelength of approximately 800 nm. The control unit first causes the second light source to emit the second measurement light, and causes the scanning mirror to scan the second measurement light to irradiate the subject's fundus with the second measurement light. The control unit acquires an image of the subject's fundus from the second measurement light reflected from the fundus and identifies the location of retinal blood vessels from the fundus image. Subsequently, the control unit drives the scanning mirror to stop it at a position where the first measurement light irradiates the identified retinal blood vessels. The control unit then causes the first light source to emit the first measurement light and irradiates the identified retinal blood vessels with the first measurement light. More specifically, the control unit changes the wavelength of the laser light irradiated onto the identified retinal blood vessels between 900 nm and 1200 nm over time. The control unit obtains information on the subject's blood glucose level based on the light intensity spectrum of the first measurement light reflected from the retinal blood vessels and the light intensity spectrum of the first measurement light.

However, the measurement apparatus described in JP2021-36938A acquires a fundus image by scanning a second measurement light (infrared laser light) with the scanning mirror, identifies the location of retinal blood vessels from the fundus image, and drives the scanning mirror to stop it at a position where the first measurement light is irradiated onto the identified retinal blood vessels. Subsequently, the measurement apparatus described in JP2021-36938A irradiates the retinal blood vessels with the first measurement light (laser light whose wavelength changes over time). As a result, the imaging duration is relatively long in the measurement apparatus described in JP2021-36938A.

### SUMMARY

The present disclosure provides an examining system, an image processing system, a non-transitory computer-readable storage medium, a wearable device, and an examining method.

An examining system according to one aspect of the present disclosure is an examining system for examining biometric information of a subject. The examining system includes an imaging unit, an image processing circuit, and a determination processing circuit. The imaging unit includes an image sensor. The imaging unit simultaneously captures, by the image sensor, light components in more than three wavelength ranges contained in light reflected from a fundus of the subject to generate image information used to determine the biometric information of the subject. The image processing circuit generates an image of the fundus for each wavelength range from the image information. The determination processing circuit determines the biometric information of the subject based on the image of the fundus for each wavelength range.

An image processing system according to one aspect of the present disclosure is an image processing system for examining biometric information of a subject. The image processing system includes an interface, an image processing circuit, and a determination processing circuit. The interface acquires image information used to determine the biometric information of the subject from an imaging unit. The imaging unit simultaneously captures light components in more than three wavelength ranges contained in light reflected from a fundus of the subject to generate the image information. The image processing circuit generates an image of the fundus for each wavelength range from the image information. The determination processing circuit determines the biometric information of the subject based on the image of the fundus for each wavelength range.

A non-transitory computer-readable storage medium according to one aspect of the present disclosure is a non-transitory computer-readable storage medium storing an examining program for examining biometric information of a subject. The program causes a computer to execute processes including:
acquiring image information used to determine the biometric information of the subject from an imaging unit, the imaging unit simultaneously capturing light components in more than three wavelength ranges contained in light reflected from a fundus of the subject to generate the image information;
generating an image of the fundus for each wavelength range from the image information; and
determining the biometric information of the subject based on the image of the fundus for each wavelength range.

A wearable device according to one aspect of the present disclosure is a wearable device attached to the head of a subject. The wearable device includes an imaging unit and a light-shielding portion. The imaging unit includes an image sensor and simultaneously captures, by the image sensor, light components in more than three wavelength ranges contained in light reflected from a fundus of the subject to generate image information used to determine the biometric information of the subject. The light-shielding portion shields an area around at least one eye of the subject.

An examining method according to one aspect of the present disclosure is an examining method for examining biometric information of a subject. The examining method includes:
imaging a fundus of the subject using an image sensor to generate image information used to determine the biometric information of the subject, the image sensor simultaneously capturing light components in more than three wavelength ranges contained in incident light;
generating, by an image processing circuit, an image of the fundus for each wavelength range from the image information; and
determining, by a determination processing circuit, the biometric information of the subject based on the image of the fundus for each wavelength range.

### BRIEF DESCRIPTION OF DRAWINGS

FIG. 1 shows an example of a wearable device included in an examining system according to a first embodiment of the present disclosure.

FIG. 2A shows the configuration of a first example of the wearable device included in the examining system according to the first embodiment of the present disclosure.

FIG. 2B shows an example of a light intensity spectrum of light emitted from a halogen lamp.

FIG. 3A shows a configuration of a second example of the wearable device included in the examining system according to the first embodiment of the present disclosure.

FIG. 3B shows an example of a light intensity spectrum of light emitted from an LED.

FIG. 4 shows the examining system according to the first embodiment of the present disclosure.

FIG. 5 is a schematic diagram illustrating a configuration of a hyperspectral filter.

FIG. 6 shows a relationship between a target wavelength band and a plurality of wavelength ranges included in the target wavelength band.

FIG. 7 shows an example of a light transmission spectrum of one filter included in the hyperspectral filter.

FIG. 8 shows an example of a light transmittance of the hyperspectral filter.

FIG. 9 shows an example of fundus images for respective wavelength ranges generated from a compressed image.

FIG. 10 is a flowchart showing a flow of processing performed by a controller included in an imaging apparatus before capturing images of the fundus of a user.

FIG. 11 is a flowchart showing a flow of an examining method according to the first embodiment of the present disclosure.

FIG. 12 shows an examining system according to a first variant.

FIG. 13 shows an examining system according to a second variant.

FIG. 14 shows an examining system according to a third variant.

FIG. 15 shows an examining system according to a fourth variant.

FIG. 16 shows an examining system according to a fifth variant.

### DETAILED DESCRIPTION

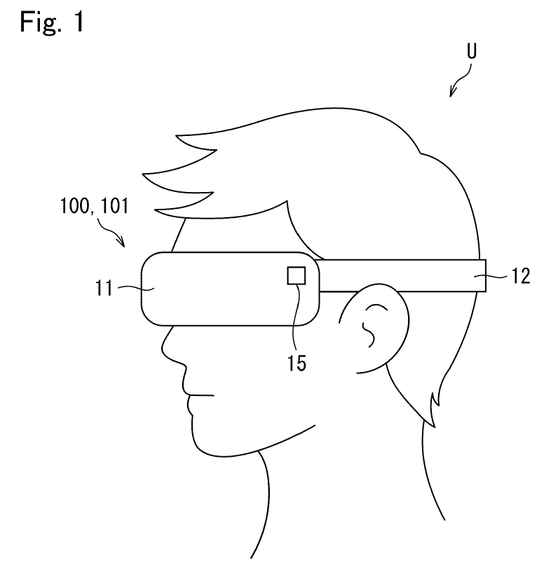

Embodiments of an examining system, an image processing system, a non-transitory computer-readable storage medium, a wearable device, and an examining method according to the present disclosure will now be described with reference to the drawings (FIGS. 1-16). However, this disclosure is not limited to the embodiments described below, and can be implemented in various manners without departing from the gist. In the figures, the same or corresponding parts are denoted by the same reference numerals, and explanations may be omitted or simplified as appropriate where explanations would otherwise be repeated.

### First Embodiment

### 1. Configuration

### 1-1. Wearable Device

FIG. 1 shows an example of a wearable device 101 included in an examining system 100 according to a first embodiment of the present disclosure. The examining system 100 examines biometric information of a user U. The user U is a subject whose biometric information is to be examined. In the first embodiment, the biometric information includes the blood glucose level. The examining system 100 examines the blood glucose level of the user U.

As shown in FIG. 1, the examining system 100 includes the wearable device 101. The wearable device 101 is worn on the head of the user U. The wearable device 101 may be, for example, a goggle type. In the first embodiment, the wearable device 101 has a light-shielding portion 11 and a belt 12.

### 1-2. Light-Shielding Portion

The light-shielding portion 11 shields the area around eyes E of the user U. More specifically, the light-shielding portion 11 is positioned on the front of the user U's head and shields the area around both eyes E of the user U. The light-shielding portion 11 extends transversely along the front of the user U's head. For example, the light-shielding portion 11 may extend along the front of the user U's head from a right end to a left end of the front of the user U’s head. Alternatively, the light-shielding portion 11 may extend along the front of the user U's head from one temporal region of the head to the other temporal region of the head.

### 1-3. Belt

The belt 12 holds the wearable device 101 on the user U's head so that the light-shielding portion 11 is positioned on the front of the user U's head. More specifically, the belt 12 may be connected to the light-shielding portion 11 and may extend in a U-shape. In other words, the belt 12 may form an annular shape together with the light-shielding portion 11. For example, one end and the other end of the belt 12 may be connected to the light-shielding portion 11. In a configuration in which one end and the other end of the belt 12 are connected to the light-shielding portion 11, the belt 12 extends from one end of the light-shielding portion 11 through the back of the user U's head to the other end of the light-shielding portion 11.

### 1-4. First Example of Wearable Device

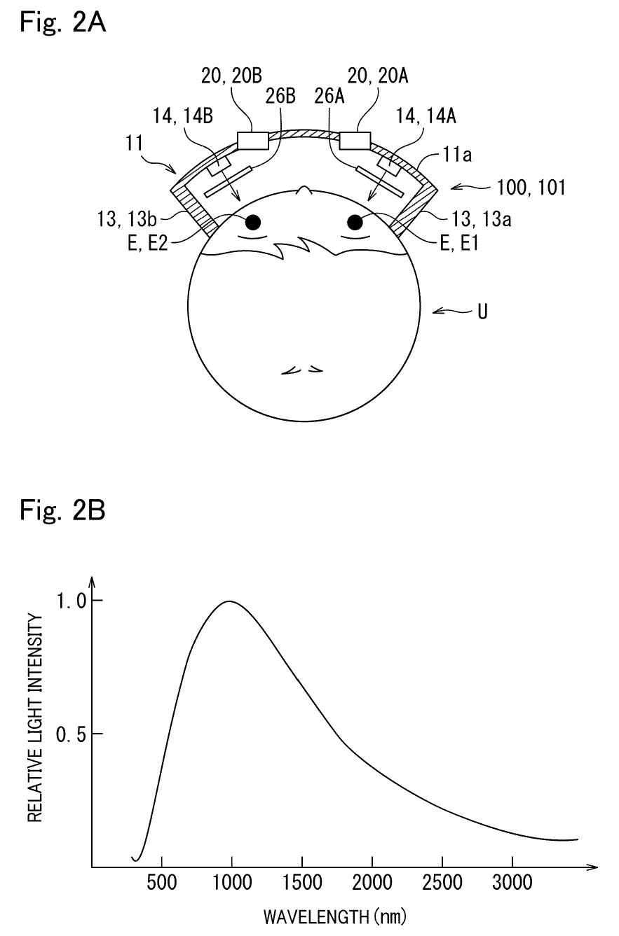

Next, a first example of the wearable device 101 will be described with reference to FIGS. 2A and 2B. FIG. 2A is a diagram showing a configuration of the first example of the wearable device 101 included in the examining system 100 according to the first embodiment of this disclosure. Specifically, FIG. 2A shows a cross-sectional view of the wearable device 101 as seen from above the user U's head. Note that the belt 12 shown in FIG. 1 is omitted in FIG. 2A.

As shown in FIG. 2A, the wearable device 101 further comprises a first imaging unit 20A, a second imaging unit 20B, a first light source 14A, and a second light source 14B. The light-shielding portion 11 has a front wall portion 11a, a first support portion 13a, and a second support portion 13b.

### 1-4-1. Front Wall Portion

The front wall portion 11a constitutes a front wall of the light-shielding portion 11. The front wall portion 11a faces the front of the user U's head. More specifically, the front wall portion 11a faces a right eye E1 and a left eye E2 of the user U. For example, the front wall portion 11a may extend along the front of the user U's head from the right end to the left end of the front of the user U's head. Alternatively, the front wall portion 11a may extend along the front of the user U's head from one temporal region of the head to the other temporal region of the head.

### 1-4-2. Support Portion

The first support portion 13a and the second support portion 13b are connected to the front wall portion 11a and support the front wall portion 11a. The first support portion 13a and the second support portion 13b constitute side walls of the light-shielding portion 11. Specifically, the first support portion 13a protrudes from the front wall portion 11a toward a part of the front of the user U's head located on a right side of the right eye E1. For example, the first support portion 13a may protrude from one end of the front wall portion 11a. Similarly, the second support portion 13b protrudes from the front wall portion 11a toward a part of the front of the user U's head located on a left side of the left eye E2. For example, the second support portion 13b may protrude from the other end of the front wall portion 11a. Hereafter, when it is not necessary to distinguish between the first support portion 13a and the second support portion 13b, the first support portion 13a and the second support portion 13b may be referred to as "support portion 13".

### 1-4-3. Imaging Unit

The first imaging unit 20A is supported by the front wall portion 11a. The first imaging unit 20A images the fundus of the user U's right eye E1. Therefore, the subject image captured by the first imaging unit 20A is an image of the fundus of the user U's right eye E1. For example, the first imaging unit 20A is positioned directly in front of the user U's right eye E1.

More specifically, the first imaging unit 20A simultaneously captures light components in more than three wavelength ranges contained in light reflected from the fundus of the user U's right eye E1, and generates image information used to determine biometric information of the user U. More specifically, the first imaging unit 20A generates two-dimensional image data in which fundus images for respective wavelength ranges are superimposed as image information used to determine biometric information of the user U.

The second imaging unit 20B is supported by the front wall portion 11a and images the fundus of the user U's left eye E2. The configuration of the second imaging unit 20B is the same as that of the first imaging unit 20A, so that a detailed explanation is omitted.

In the following explanation, when it is not necessary to distinguish between the first imaging unit 20A and the second imaging unit 20B, both the first imaging unit 20A and the second imaging unit 20B may be referred to simply as "imaging unit 20".

A distance between the user U's right eye E1 and the first imaging unit 20A, and a distance between the user U's left eye E2 and the second imaging unit 20B are maintained at a first predetermined distance by the support portion 13. The first predetermined distance is, for example, 5 cm or more and 10 cm or less.

According to the first embodiment, the area around the user U's eye E is shielded from light by the light-shielding portion 11, allowing the imaging unit 20 to more reliably image the fundus of the user U. Specifically, if visible light from the surrounding environment reaches the user U's eye E, the user U's pupil may constrict, making it impossible to image the fundus of the user U. In contrast, according to the first embodiment, the area around the user U's eye E is shielded from light by the light-shielding portion 11. As a result, it becomes more difficult for visible light from the surrounding environment to reach the user U's eye E, and the user U's pupil becomes more likely to dilate. Therefore, the imaging unit 20 more reliably images the fundus of the user U.

Furthermore, according to the first embodiment, since the imaging unit 20 is positioned in front of the user U's eye E, the imaging unit 20 more reliably images the fundus of the user U.

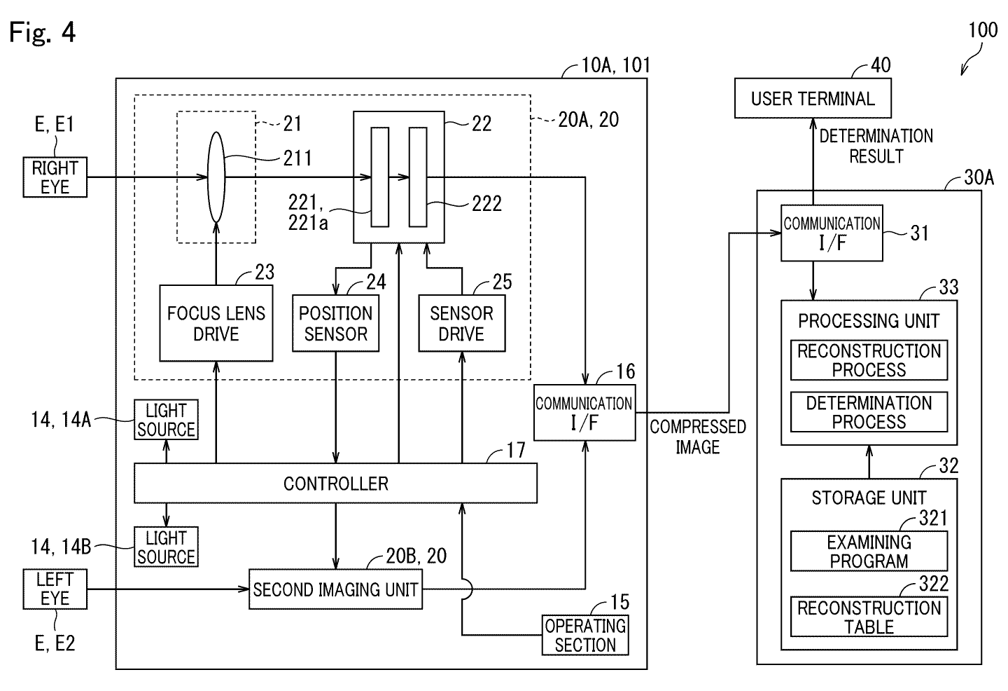

Furthermore, according to the first embodiment, the support portion 13 maintains the first predetermined distance between the imaging unit 20 and the user U's eye E, thereby facilitating focusing on the fundus of the eye E. More specifically, by maintaining the first predetermined distance between the imaging unit 20 and the eye E, the optical distance between an imaging device 22 (described later with reference to FIG. 4) and the eye E is maintained at a second predetermined distance. As a result, a focusing process on the fundus of the eye E is facilitated.

### 1-4-4. Light Source

The first light source 14A and the second light source 14B are supported by the front wall portion 11a. Light emitted from the first light source 14A irradiates the fundus of the user U's right eye E1. A direction in which the light is emitted from the first light source 14A is approximately the same as a direction from the first light source 14A toward the right eye E1. Light emitted from the second light source 14B also irradiates the fundus of the user U's left eye E2, similar to the light emitted from the first light source 14A. Hereinafter, the light emitted from the first light source 14A may be referred to as "first light". Similarly, the light emitted from the second light source 14B may be referred to as "second light". Furthermore, when it is not necessary to distinguish between the first light source 14A and the second light source 14B, the first light source 14A and the second light source 14B may be referred to as "light source 14".

The light source 14 emits light having wavelengths necessary to acquire biometric information of an object to be examined. For example, if the object to be examined is blood glucose level, the first and second lights include infrared light. Therefore, the light reflected from the user U's fundus and incident on the imaging unit 20 includes infrared light. More specifically, if the object to be examined is blood glucose level, the first and second lights include short-wave infrared light. Therefore, the light reflected from the user U's fundus and incident on the imaging unit 20 includes short-wave infrared light. However, the first and second lights may include near-infrared light, or near-infrared light and mid-infrared light may be included. In the first embodiment, the first and second lights further include visible light.

Infrared light has a longer wavelength than visible light. Specifically, the lower limit of the wavelength range of visible light is approximately 360 nm to 400 nm. The upper limit of the wavelength range of visible light is approximately 760 nm to 830 nm. The wavelength range of infrared light is approximately 780 nm to 1 mm. The wavelength range of short-wave infrared light is approximately 1000 nm to 1700 nm. The wavelength range of near-infrared light is approximately 780 nm to 2500 nm. The wavelength range of mid-infrared light is approximately 2500 nm to 4000 nm.

An illuminance of the first and second lights is set to a value that the user can perceive and that does not cause the user's pupils to constrict. For example, the illuminance of the first and second lights is greater than or equal to 10 lux and less than 500 lux. A lower limit of the illuminance of the first and second lights is set to the lower limit of illuminance that a person can perceive as light. If the illuminance of the first and second lights is around 10 lux, the user can perceive the first and second lights as a dim light. An upper limit of the illuminance of the first and second lights is set such that the person's pupils do not constrict. If the illuminance exceeds 500 lux, the user's pupils may constrict.

### 1-4-5. Guide Light

In the first embodiment, the light source 14 functions as a guide light source that emits guide light to direct the line of sight of the user U toward the imaging unit 20. Specifically, the first light functions as a guide light to direct the line of sight of the user's right eye E1 toward the first imaging unit 20A. The second light functions as a guide light to direct the line of sight of the user's left eye E2 toward the second imaging unit 20B.

Specifically, the first light source 14A is positioned such that when the line of sight of the user U's right eye E1 is directed toward the first light source 14A, the line of sight of the user U's right eye E1 is directed approximately toward the front of the first imaging unit 20A. The second light source 14B is positioned similarly to the first light source 14A.

Note that in FIG. 2A, the dimensions of the imaging unit 20 and the light source 14 are shown enlarged for ease of understanding, but the dimensions of the imaging unit 20 and the light source 14 shown are different from the actual dimensions. Also, in FIG. 2A, the light source 14 is shown at a position spaced apart from the imaging unit 20 for ease of understanding, but the position of the light source 14 shown is different from the actual position. The first light source 14A may be positioned closer to the first imaging unit 20A than the position shown. Similarly, the second light source 14B may be positioned closer to the second imaging unit 20B than the position shown. However, the position of the first light source 14A is not particularly limited, as long as the first light functions as a guide light that directs the line of sight of the user's right eye E1 toward the first imaging unit 20A. The same applies to the position of the second light source 14B.

According to the first embodiment, the light source 14 direct the line of sight of the user toward the imaging unit 20. Therefore, the user U's fundus is imaged more reliably.

### 1-4-6. Diffuser

The wearable device 101 may further include a transmissive diffuser plate 26A positioned between the first light source 14A and the user U's right eye E1, and a transmissive diffuser plate 26B positioned between the second light source 14B and the user U's left eye E2.

The diffusers 26A and 26B are optical elements that have light diffusion characteristics with low wavelength dependence and low directional dependence within the wavelength bands of the first and second lights. For example, the diffusers 26A and 26B may be frosted glass or translucent plates made of resin. The translucent plate is an optical element that diffuses light as it passes through the translucent plate.

The diffuser plate 26A diffuses the light emitted from the first light source 14A. Similarly, the diffuser plate 26B diffuses the light emitted from the second light source 14B. As a result, the user U's eye E is illuminated by the light diffused through the diffuser plates 26A and 26B. By disposing the diffuser plates 26A and 26B, the fundus of each of the user U's right eye E1 and left eye E2 is uniformly illuminated by the first and second lights. As a result, the user U's fundus is imaged more reliably.

The diffuser plate 26A is positioned, for example, in the vicinity of the first light source 14A. Alternatively, the diffuser plate 26A may be in contact with the first light source 14A. For example, the diffuser plate 26A may be fixed to the front wall portion 11a or the first light source 14A by fastening members such as screws. Alternatively, the diffuser plate 26A may be supported by a structure extending from the front wall portion 11a or the first light source 14A toward the front of the user U's head. Similarly, the diffuser plate 26B may be positioned near the second light source 14B or in contact with the second light source 14B. By positioning the diffuser plate 26A near the first light source 14A or in contact with the first light source 14A, the light emitted from the first light source 14A is diffused more sufficiently. Similarly, by positioning the diffuser plate 26B near the second light source 14B or in contact with the second light source 14B, the light emitted from the second light source 14B is diffused more sufficiently. Therefore, the fundus of each of the user U's right eye E1 and left eye E2 is illuminated more uniformly. As a result, user U's fundus is imaged more reliably.

### 1-4-7. Halogen Lamp

Referring then to FIG. 2B, an example of the first light source 14A and the second light source 14B will be described. Each of the first light source 14A and the second light source 14B may be, for example, a halogen lamp. FIG. 2B is a diagram showing an example of a light intensity spectrum of light emitted from a halogen lamp. In FIG. 2B, the horizontal axis represents a wavelength of light emitted from a halogen lamp. The vertical axis represents a relative light intensity of the light emitted from a halogen lamp. Hereinafter, the light emitted from a halogen lamp may be referred to as "halogen light".

As illustrated in FIG. 2B, a light intensity spectrum of halogen light may have a shape that rises from around 500 nm, peaks around 1000 nm, and has a long tail extending from the peak toward longer wavelengths.

As illustrated in FIG. 2B, halogen light includes short-wave infrared light. Therefore, by using halogen lamps as the first light source 14A and the second light source 14B, the blood glucose level of the user U can be examined. Also, as illustrated in FIG. 2B, halogen light includes visible light. Therefore, the first and second lights can be used as guide lights.

### 1-5. Second Example of Wearable Device

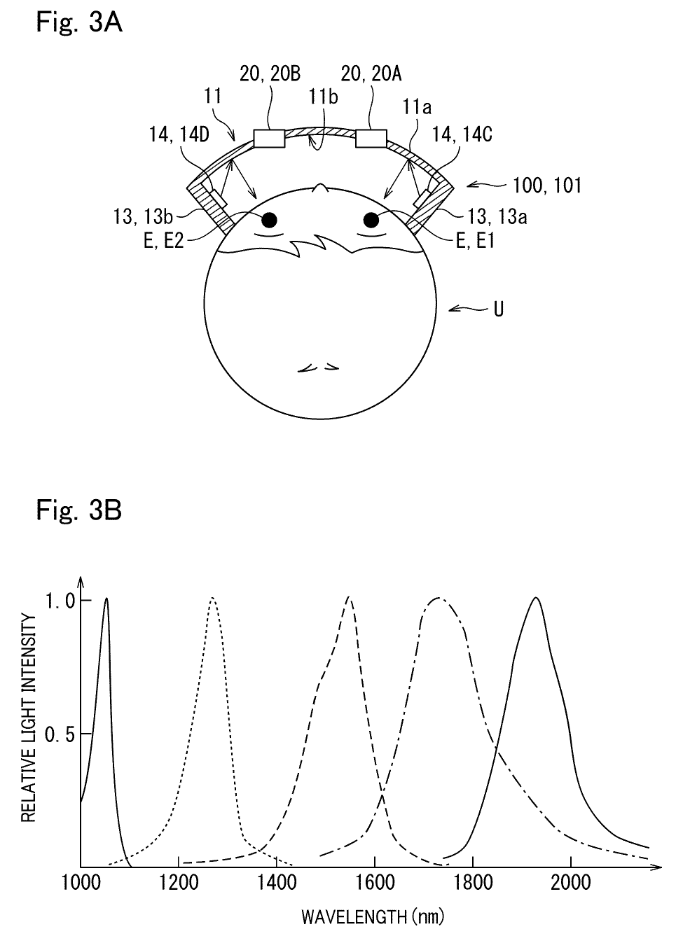

Next, a second example of the wearable device 101 will be described with reference to FIGS. 3A and 3B. However, the following description focuses on differences from the first example of the wearable device 101 described with reference to FIGS. 2A and 2B, and redundant descriptions may be omitted.

FIG. 3A shows a configuration of the second example of the wearable device 101 included in the examining system 100 according to the first embodiment of the present disclosure. More specifically, FIG. 3A shows a cross-sectional view of the wearable device 101 as seen from above the user U's head. Note that the belt 12 shown in FIG. 1 is omitted in FIG. 3A.

As shown in FIG. 3A, in the second example of the wearable device 101, a third light source 14C and a fourth light source 14D are provided in the wearable device 101, instead of the first light source 14A and the second light source 14B described with reference to FIGS. 2A and 2B.

### 1-5-1. Light Source

The third light source 14C is supported, for example, by the first support portion 13a. The fourth light source 14D is supported, for example, by the second support portion 13b. The third light source 14C emits light toward an inner wall surface 11b of the front wall portion 11a. Similarly, the fourth light source 14D emits light toward the inner wall surface 11b of the front wall portion 11a. The inner wall surface 11b is the surface facing the front of the user U's head. Hereinafter, the light emitted from the third light source 14C may be referred to as "third light". Similarly, the light emitted from the fourth light source 14D may be referred to as "fourth light".

### 1-5-2. Inner Wall Surface of Front Wall Portion

The inner wall surface 11b of the front wall portion 11a reflects the third light toward the user U's right eye E1. As a result, the third light is irradiated onto the fundus of the user U's right eye E1. A direction in which light is emitted from the third light source 14C is adjusted so that the third light is irradiated onto the fundus of the user U's right eye E1. A shape of the area on the inner wall surface 11b where the third light is reflected is adjusted so that the third light is irradiated onto the fundus of the user U's right eye E1. Hereafter, the direction in which light is emitted from the third light source 14C may be referred to as the "light emission direction of the third light source 14C". Also, the area on the inner wall surface 11b where the third light is reflected may be referred to as "first reflection site on the inner wall surface 11b".

Similarly, the inner wall surface 11b of the front wall portion 11a reflects the fourth light toward the user U's left eye E2. A direction from which light is emitted from the fourth light source 14D is adjusted so that the fourth light is directed toward the fundus of the user U's left eye E2. A shape of the area on the inner wall surface 11b where the fourth light is reflected is adjusted so that the fourth light is directed toward the fundus of the user U's left eye E2. Hereafter, the direction from which light is emitted from the fourth light source 14D may be referred to as the "light emission direction of the fourth light source 14D". Also, the area on the inner wall surface 11b where the fourth light is reflected may be referred to as "second reflection site on the inner wall surface 11b".

### 1-5-3. Wavelength Band and Illuminance

The wavelength ranges and illuminances of the third and fourth lights are the same as those of the first and second lights, so that their explanation will be omitted.

Also in describing the second example of the wearable device 101, when it is not necessary to distinguish between the third light source 14C and the fourth light source 14D, the third light source 14C and the fourth light source 14D may be referred to simply as "light source 14".

### 1-5-4. Guide Light

In the second example of the wearable device 101, the light source 14 may function as a guide light source that emits guide light to direct the line of sight of the user U toward the imaging unit 20. Specifically, the third light may function as a guide light to direct the line of sight of the user's right eye E1 toward the first imaging unit 20A. The fourth light may function as a guide light to direct the line of sight of the user's left eye E2 toward the second imaging unit 20B. Specifically, the light emission direction of the third light source 14C and the shape of the first reflection site on the inner wall surface 11b may be adjusted so that the line of sight of the user U's right eye E1 is directed toward the first reflection site on the inner wall surface 11b, thereby directing the line of sight of the user U's right eye E1 toward approximately the front of the first imaging unit 20A. The same applies to the light emission direction of the fourth light source 14D and the shape of the second reflection site on the inner wall surface 11b.

Note that in FIG. 3A, the dimensions of the imaging unit 20 and the light source 14 are shown enlarged for ease of understanding, but the dimensions of the imaging unit 20 and the light source 14 shown are different from the actual dimensions. Also, in FIG. 3A, the position of the first reflection site on the inner wall surface 11b is shown to be far from the first imaging unit 20A for ease of understanding, but the position of the first reflection site shown is different from the actual position. When the third light is used as a guide light, the third light is reflected at a position closer to the first imaging unit 20A than the position of the first reflection site shown. Similarly, when the fourth light is used as a guide light, the fourth light is reflected at a position closer to the second imaging unit 20B than the position of the second reflection site shown. However, the position of the first reflection site on the inner wall surface 11b is not particularly limited, as long as it is a position in which the third light functions as a guide light that directs the line of sight of the user's right eye E1 toward the first imaging unit 20A. The same applies to the position of the second reflection site on the inner wall surface 11b.

### 1-5-5. Light Diffusion

In the second example of the wearable device 101, the inner wall surface 11b of the front wall portion 11a diffuses and reflects the third and fourth lights. Therefore, the fundus of the user U is uniformly illuminated with the third and fourth lights. As a result, the fundus of the user U is imaged more reliably.

For example, by using barium sulfate or the like as a material for the inner wall surface 11b of the front wall portion 11a, the third and fourth lights are diffused and reflected at the inner wall surface 11b of the front wall portion 11a. Alternatively, the inner wall surface 11b of the front wall portion 11a may be roughened so that the third and fourth lights are diffused and reflected. In a configuration in which the inner wall surface 11b is roughened, a material for the inner wall surface 11b of the front wall portion 11a may be, for example, resin or ceramic.

### 1-5-6. Light Emitting Diode (LED)

Referring then to FIG. 3B, an example of the third light source 14C and the fourth light source 14D will be described. FIG. 3B is a diagram showing an example of a light intensity spectrum of light emitted from an LED. More specifically, FIG. 3B shows an example of light intensity spectra of lights emitted from five infrared LEDs with different wavelength bands. An infrared LED is an LED that emits infrared light. In FIG. 3B, the horizontal axis represents a wavelength of light emitted from an infrared LED. The vertical axis represents a relative light intensity of the light emitted from an infrared LED. Hereinafter, the light emitted from an LED may be referred to as "LED light". As shown in FIG. 3B, the full width at half maximum of LED light is typically between 50 nm and 200 nm. The five LED lights with different wavelength bands have different peak wavelengths.

In the second example of the wearable device 101, each of the third light source 14C and the fourth light source 14D has a plurality of LEDs with different wavelength bands. Each of the third light source 14C and the fourth light source 14D simultaneously emits a plurality of LED lights with different wavelength bands. For example, each of the third light source 14C and the fourth light source 14D may simultaneously emit five LED lights (infrared light) as illustrated in FIG. 3B. As illustrated in FIG. 3B, the third light may include LED lights. Among the LED lights of the third light, light intensity spectra of two LED lights having adjacent peak wavelengths may partially overlap. The same applies to the fourth light.

More specifically, in the second example of the wearable device 101, each of the third light source 14C and the fourth light source 14D has a plurality of LEDs selected to emit light having wavelengths necessary for acquiring biometric information of an object to be examined. When the object to be examined is blood glucose levels, each of the third light source 14C and the fourth light source 14D has a plurality of infrared LEDs with different wavelength bands. More specifically, when the object to be examined is blood glucose levels, each of the third light source 14C and the fourth light source 14D has a plurality of short-wave infrared LEDs with different wavelength bands. A short-wave infrared LED is an LED that emits short-wave infrared light. However, each of the third light source 14C and the fourth light source 14D may also have a plurality of near-infrared LEDs with different wavelength bands. A near-infrared LED is an LED that emits near-infrared light.

In the first embodiment, each of the third light source 14C and the fourth light source 14D further includes a visible light LED. The visible light LED is an LED that emits visible light. By having a visible light LED in each of the third light source 14C and the fourth light source 14D, the third light source 14C and the fourth light source 14D function as guide light sources.

The wearable device 101 may also have two additional guide light sources in addition to the third light source 14C and the fourth light source 14D. The guide light sources may be, for example, visible light LEDs. The two guide light sources may be arranged in the same positions as the first light source 14A and the second light source 14B, as described with reference to FIGS. 2A and 2B. If the wearable device 101 has two additional guide light sources in addition to the third light source 14C and the fourth light source 14D, the third light source 14C and the fourth light source 14D do not need to function as guide light sources. Therefore, in this case, the third light source 14C and the fourth light source 14D need not include visible light LEDs.

### 1-6. Examining System

Next, the examining system 100 of the first embodiment will be described with reference to FIG. 4. FIG. 4 is a diagram showing the examining system 100 according to the first embodiment of the present disclosure. Specifically, FIG. 4 shows a configuration of an imaging apparatus 10A included in the wearable device 101 and a configuration of an image processing system 30A included in the examining system 100.

### 1-7. Imaging Apparatus

As shown in FIG. 4, the wearable device 101 includes the imaging apparatus 10A for imaging the fundus of the user U. The imaging apparatus 10A includes a light source 14, an operating section 15, a communication interface 16, a controller 17, the first imaging unit 20A, and the second imaging unit 20B. In the example shown in FIG. 4, the imaging apparatus 10A has the first light source 14A and the second light source 14B as the light source 14, as described with reference to FIGS. 2A and 2B. The imaging apparatus 10A may also have the third light source 14C and the fourth light source 14D, as described with reference to FIGS. 3A and 3B, instead of the first light source 14A and the second light source 14B.

The imaging apparatus 10A is a spectral camera. In the first embodiment, the imaging apparatus 10A is a hyperspectral camera. The imaging apparatus 10A images the fundus of the user U and generates image information used to determine biometric information of the user U. Specifically, the imaging apparatus 10A simultaneously captures light components in more than three wavelength ranges contained in the light reflected from the user U's fundus, and generates two-dimensional image data in which fundus images for wavelength ranges are superimposed. In the first embodiment, the two-dimensional image data is compressed image data. Compressed image data will be described later. Note that more than three wavelength ranges mean, for example, more wavelength ranges than the three wavelength ranges of red, green, and blue acquired by a standard color camera.

### 1-8. Operating Section

The operating section 15 is a user interface that receives operations from the user U. The operating section 15 includes at least one of a hard key and a software key. For example, the operating section 15 includes at least one of a button, a mode dial, a touch panel, and a switch. When the operating section 15 receives an operation from the user U, the operating section 15 transmits an operation signal corresponding to the operation to the controller 17. In the first embodiment, as shown in FIG. 1, the operating section 15 is provided in the light-shielding portion 11. The operating section 15 may be located, for example, near the user U's temple.

Specifically, the mode dial accepts a mode selection operation from the user U. For example, the mode dial accepts an operation to switch an imaging mode between a mode for imaging the right eye E1 and a mode for imaging the left eye E2.

The button includes a shutter button. Based on an operation of the user U received by the shutter button, the controller 17 causes the first imaging unit 20A or the second imaging unit 20B to image the fundus of the user U.

### 1-9. Communication Interface

The communication interface 16 transmits image information used to determine the biometric information of the user U to the image processing system 30A. More specifically, the communication interface 16 transmits two-dimensional image data to the image processing system 30A. The communication interface 16 is an example of an "output unit". Specifically, the communication interface 16 performs data communication in accordance with existing wired communication standards or existing wireless communication standards. The communication interface 16 is connectable to a network. The communication interface 16 sends and receives information with external apparatuses via a network. Networks include intranets, the Internet, and the like. Alternatively, the communication interface 16 may communicate directly with external apparatuses without going through a network. When the communication interface 16 communicates directly with external apparatuses without going through a network, the communication interface 16 may perform communication in accordance with standards such as a universal serial bus (USB), HDMI (registered trademark), Bluetooth (registered trademark), etc.

### 1-10. Controller

The controller 17 provides overall control over the operation of the entire imaging apparatus 10A. For example, the controller 17 controls the light source 14 to emit light. The controller 17 controls the imaging unit 20 to image the fundus. The controller 17 controls the communication interface 16 to transmit image information from the communication interface 16 to the image processing system 30A.

The controller 17 may include electronic circuits configured to implement predetermined functions by executing a computer program. For example, the controller 17 may have one or more processors. The one or more processors include a central processing unit (CPU), a micro processing unit (MPU), a graphics processing unit (GPU), a data processing unit (DPU), a digital signal processor (DSP), a field programmable gate array (FPGA), an application specific integrated circuit (ASIC), and the like.

Furthermore, the controller 17 may include a memory. The memory stores computer programs executed by the one or more processors. Furthermore, the memory stores configuration information that the one or more processors refers to when executing computer programs. The memory is, for example, semiconductor memory. Specifically, the controller 17 may include at least one of a read only memory (ROM) and a random access memory (RAM). Alternatively, the controller 17 may include at least one of a flash memory, an erasable programmable read only memory (EPROM), and an electrically erasable programmable read only memory (EEPROM) instead of, or in addition to, RAM and ROM.

### 1-11. First Imaging Unit

As shown in FIG. 4, the first imaging unit 20A includes an image-forming optical system 21 and the imaging device 22. In the first embodiment, the first imaging unit 20A further includes a focusing lens driving unit 23, a position sensor 24, and a sensor driving unit 25.

### 1-11-1. Image-Forming Optical System

The image-forming optical system 21 is positioned in front of the user U's right eye E1. The image-forming optical system 21 causes the light reflected from the fundus of the user U's right eye E1 to form an image on the imaging device 22. More specifically, the image-forming optical system 21 causes the fundus image to form on an image sensor 222 included in the imaging device 22. The image-forming optical system 21 includes one or more lenses. For example, the image-forming optical system 21 may include a focusing lens 211.

The focusing lens 211 changes the focused state of a subject image formed on the imaging device 22. The subject image is the fundus image of the user U's right eye E1. The focusing lens 211 is composed of one or more lenses. The focusing lens 211 is driven by the focusing lens driving unit 23.

Furthermore, the image-forming optical system 21 may include, in addition to the focusing lens 211, a zoom lens, an optical image stabilization lens, an aperture, and a shutter. For example, the zoom lens changes the magnification of the subject image formed by the image-forming optical system 21. By disposing a zoom lens in the image-forming optical system 21, it is possible to magnify and image retinal blood vessels RV of the fundus of the eye E.

### 1-11-2. Focusing Lens Driving unit

The focusing lens driving unit 23 is controlled by the controller 17 to move at least one of the one or more lenses of the focusing lens 211 along the optical axis of the image-forming optical system 21. The focusing lens driving unit 23 includes, for example, an actuator or motor for driving the focusing lens 211.

Furthermore, in a configuration in which the image-forming optical system 21 includes a zoom lens, the imaging unit 20 further includes a zoom lens driving unit. The zoom lens is composed of one or more lenses. The zoom lens driving unit may be controlled by the controller 17 to move at least one of the one or more lenses of the zoom lens along the optical axis direction of the image-forming optical system 21. In this case, the zoom lens driving unit may include, for example, an actuator or motor that drives the zoom lens. Alternatively, the zoom lens driving unit may have a configuration that accepts manual operation by the user U. For example, the zoom lens driving unit may include a dial member that accepts rotational operation by the user U. By rotating the dial member, at least one of the one or more lenses of the zoom lens along the optical axis direction of the image-forming optical system 21. Alternatively, the zoom lens driving unit may include a zoom ring.

### 1-11-3. Imaging Device

The imaging device 22 simultaneously captures light components in more than three wavelength ranges contained in the light reflected from the fundus of the user U's right eye E1, and generates image information used to determine biometric information of the user U. Specifically, the imaging device 22 generates two-dimensional image data in which fundus images for respective wavelength ranges are superimposed as image information used to determine biometric information of the user U. In the first embodiment, the imaging device 22 has a filter unit 221 and the image sensor 222.

The filter unit 221 is positioned in the optical path of light incident on the image sensor 222. For example, the filter unit 221 may be positioned between the image-forming optical system 21 and the image sensor 222. Light reflected from the fundus of the user U's right eye E1 is incident on the filter unit 221 via the image-forming optical system 21.

The filter unit 221 directs light within a specific wavelength range onto the image sensor 222. Hereinafter, the wavelength range of the light incident on the image sensor 222 via the filter unit 221 may be referred to as "target wavelength band λ". The target wavelength band λ is set according to biometric information of an object to be examined.

More specifically, the filter unit 221 modulates the intensity of light in the target wavelength band λ at intervals of more than three wavelength ranges and directs the modulated light onto the image sensor 222. In the first embodiment, the target wavelength band λ is included in the infrared light wavelength range, and the intensity of light in the target wavelength band λ is modulated at intervals of a predetermined number of wavelength ranges. More specifically, the target wavelength band λ is included in the short-wave infrared light wavelength range, and the intensity of light in the target wavelength band λ is modulated at intervals of a predetermined number of wavelength ranges. For example, the target wavelength band λ is between 1000 nm and 1600 nm, inclusive, and the filter unit 221 may modulate the intensity of light in the target wavelength band λ at intervals of thirty wavelength ranges.

More specifically, the filter unit 221 includes a hyperspectral filter 221a. The hyperspectral filter 221a modulates the intensity of light incident on the hyperspectral filter 221a at intervals of more than three wavelengths and outputs the modulated light. The filter unit 221 may further include at least one of an optical long-pass filter and an optical short-pass filter in order to direct light in the target wavelength band λ onto the image sensor 222.

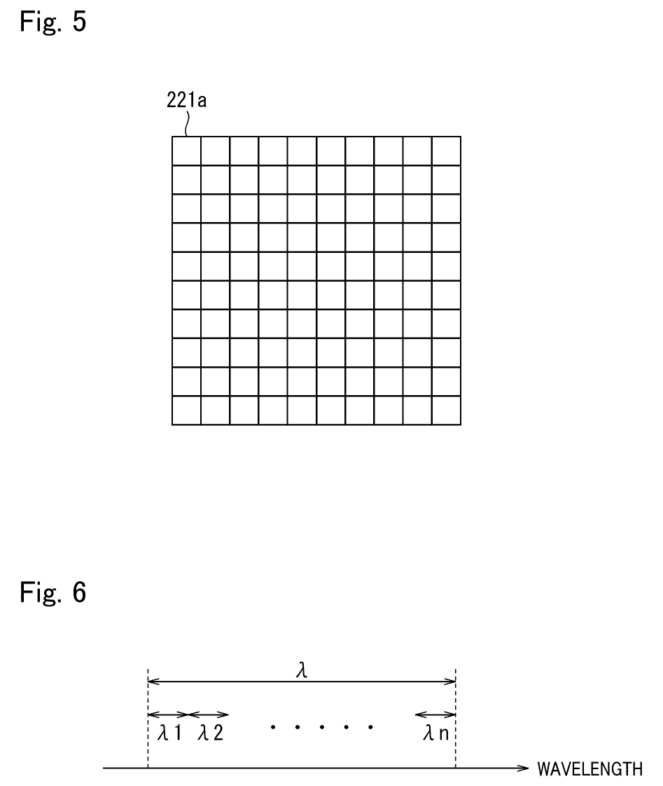

The hyperspectral filter 221a is, for example, a filter array having a plurality of optical filters arranged two-dimensionally along a plane intersecting the optical axis of the image-forming optical system 21. Typically, the filter array has a plurality of optical filters arranged two-dimensionally along a plane perpendicular to the optical axis of the image-forming optical system 21. Details of the hyperspectral filter 221a will be described later with reference to FIGS. 5 to 8.

The image sensor 222 images the fundus of the user U's right eye E1 via the image-forming optical system 21 and the filter unit 221. In other words, the image sensor 222 images the fundus image formed via the image-forming optical system 21 and the filter unit 221. More specifically, the image sensor 222 simultaneously captures light components in more than three wavelength ranges contained in the light reflected from the fundus of the user U's right eye E1. More specifically, the image sensor 222 simultaneously captures light components in more than three wavelength ranges contained in the target wavelength band λ via the filter unit 221. The filter unit 221 modulates the intensity of light in each wavelength range contained in the target wavelength band λ and directs the modulated light onto the image sensor 222. In the first embodiment, the hyperspectral filter 221a modulates the intensity of short-wave infrared light for each of thirty wavelength ranges. The image sensor 222 images the short-wave infrared light whose intensity has been modulated for each wavelength range by the hyperspectral filter 221a.

The image sensor 222 captures a fundus image and generates image information used to determine biometric information of the user. Specifically, the image sensor 222 simultaneously captures light components in more than three wavelength ranges contained in the light reflected from the fundus of the user U's right eye E1, and generates two-dimensional image data in which fundus images for respective wavelength ranges are superimposed. The timing at which the image sensor 222 generates the image information is controlled by the controller 17.

Specifically, the image sensor 222 has a plurality of pixels. The image sensor 222 may be, for example, a CMOS image sensor, a CCD image sensor, or an NMOS image sensor. The image sensor 222 captures light in the target wavelength band λ, with the intensity of the light modulated for each wavelength range by the filter unit 221. For example, the imaging device 22 may be configured so that light in the target wavelength band λ is simultaneously incident on each pixel of the image sensor 222, the intensity of the light being modulated for each wavelength range. In this case, the image sensor 222 outputs a signal from each of the plurality of pixels in which pieces of information from each wavelength range are superimposed. The image sensor 222 then uses the signals output from each of the plurality of pixels to generate image information in which the pieces of information from each wavelength range are superimposed. More specifically, the image sensor 222 generates two-dimensional image data in which the pieces of information from each wavelength range are superimposed. Therefore, the image information (two-dimensional image data) output from the image sensor 222 is compressed image data in which the pieces of information from each wavelength range are superimposed.

### 1-11-4. Configuration for Correcting Shift in Imaging Position

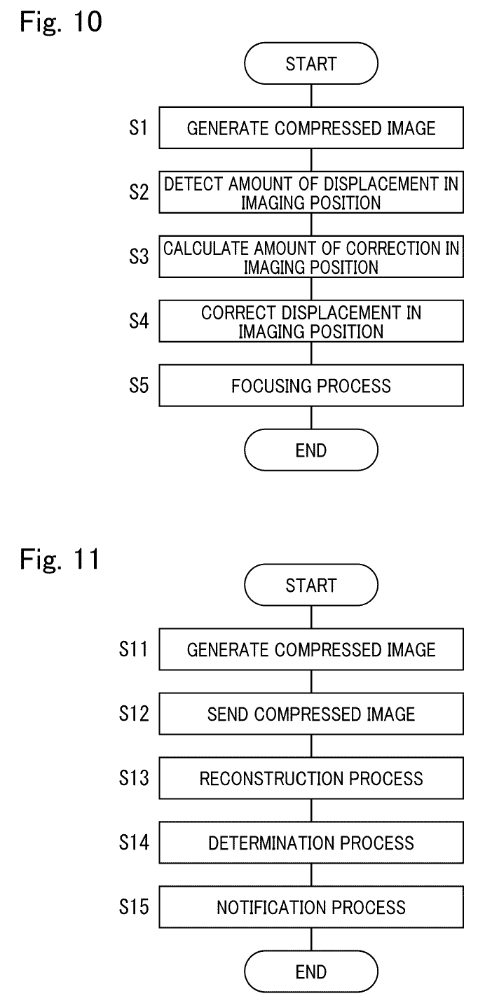

The position sensor 24 detects the current position of the imaging device 22 in a plane perpendicular to the optical axis of the image-forming optical system 21. The position sensor 24 may include, for example, a magnet and a Hall element. The sensor driving unit 25 moves the imaging device 22 in a plane perpendicular to the optical axis of the image-forming optical system 21. The sensor driving unit 25 may include, for example, an actuator or motor to move the imaging device 22. The controller 17 determines the current position of the imaging device 22 based on the signal output from the position sensor 24. The controller 17 corrects the deviation of the imaging position by controlling the sensor driving unit 25, as will be described later with reference to FIG. 10. The controller 17 is an example of a "control circuit".

### 1-12. Second Imaging Unit

A configuration of the second imaging unit 20B is substantially the same as that of the first imaging unit 20A, and hence its explanation will be omitted.

### 1-13. Image Processing System

Next, the image processing system 30A will be described. As shown in FIG. 4, in the first embodiment, the examining system 100 includes the wearable device 101 and the image processing system 30A. The image processing system 30A determines biometric information of the user U based on the image information received from the imaging apparatus 10A of the wearable device 101 and transmits the determination result of the biometric information to a user terminal 40. As shown in FIG. 4, the image processing system 30A includes a communication interface 31, a storage unit 32, and a processing unit 33. The image processing system 30A may be, for example, a general-purpose computer or a dedicated computer. Alternatively, the image processing system 30A may be a server. For example, the image processing system 30A may provide the user U with a cloud service that transmits the determination result of the biometric information to the user terminal 40.

### 1-13-1. Communication Interface

The communication interface 31 performs data communication in accordance with existing wired communication standards or existing wireless communication standards. The communication interface 31 is connectable to a network. The communication interface 31 sends and receives information with external apparatuses via a network. The network includes intranets, the Internet, and the like. Alternatively, the communication interface 31 may communicate directly with external apparatuses without going through a network. The communication interface 31 may have a configuration similar to that of the communication interface 31.

More specifically, the communication interface 31 communicates with the communication interface 16 of the imaging apparatus 10A to acquire the image information from the imaging apparatus 10A. Specifically, the communication interface 31 acquires the two-dimensional image data from the imaging apparatus 10A. The communication interface 31 also transmits the results of the determination of the biometric information of the user U to the user terminal 40.

### 1-13-2. Storage Unit

The storage unit 32 has a main memory. The main memory includes, for example, semiconductor memory. The storage unit 32 may further have an auxiliary storage apparatus. The auxiliary storage apparatus includes, for example, at least one of a solid-state drive (SSD) and a hard disk drive (HDD). The storage unit 32 may also include removable media.

The storage unit 32 stores computer programs executed by the processing unit 33, as well as configuration information that the processing unit 33 refers to when executing computer programs. More specifically, the storage unit 32 stores an examining program 321 and a reconstruction table 322.

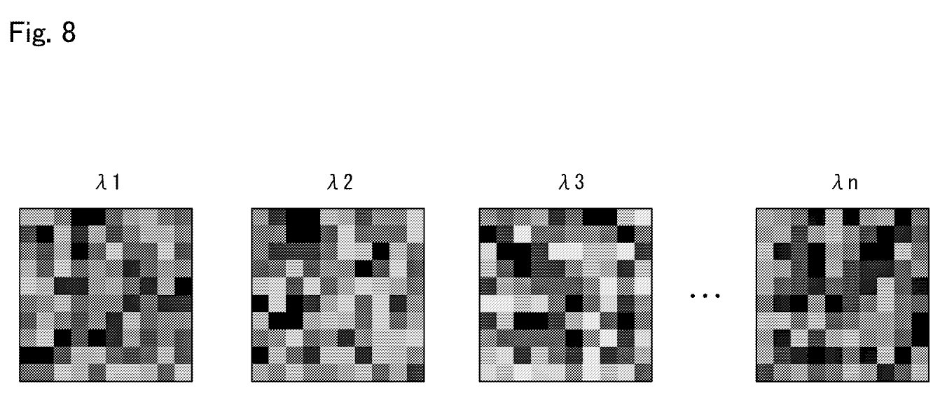

The examining program 321 causes at least one computer to execute a reconstruction process and a determination process. The reconstruction process will be described later with reference to FIG. 8. The determination process will be described later. In the first embodiment, the examining program 321 causes the processing unit 33 to execute the reconstruction process and the determination process. The examining program 321 may be stored in a computer-readable storage medium. The storage medium is a non-transitory and tangible medium. The storage medium may be provided inside or outside the computer. In the first embodiment, the examining program 321 is stored in the storage unit 32.

The examining program 321 includes a computer program that instructs the processing unit 33 to perform a reconstruction process and a computer program that instructs the processing unit 33 to perform a determination process. When the processing unit 33 performs the reconstruction process, the reconstruction table 322 is referenced by the processing unit 33. Details of the reconstruction table 322 will be described later with reference to FIG. 8.

### 1-13-3. Processing Unit

The processing unit 33 may include electronic circuits configured to implement predetermined functions by executing a computer program. For example, the processing unit 33 may have one or more processors. The one or more processors include a CPU, a MPU, a GPU, a DPU, a DSP, a FPGA, an ASIC, and the like.

The processing unit 33 executes the examining program 321 to perform a reconstruction process and generate fundus image data for each wavelength range included in the target wavelength band λ from the image information. Specifically, the image information received by the communication interface 31 from the wearable device 101 includes the two-dimensional image data. The two-dimensional image data is compressed image data in which pieces of information for respective wavelength ranges is superimposed by the hyperspectral filter 221a. The reconstruction process is a process of generating fundus images for respective wavelength ranges included in the target wavelength band λ from a compressed image. The processing unit 33 generates fundus images for respective wavelength ranges from a compressed image by referring to the reconstruction table 322. The processing unit 33 is an example of the image processing circuit.

Furthermore, the processing unit 33 executes a determination process by running the examining program 321 to determine biometric information of the user U based on the fundus images for respective wavelength ranges. For example, the processing unit 33 may acquire the intensity of light reflected from retinal blood vessels RV included in the fundus for each wavelength range based on the fundus images for respective wavelength ranges, and determine biometric information of subject based on the acquired intensity of light. The processing unit 33 is an example of a determination processing circuit. For example, based on knowledge of a correspondence between biometric information and wavelength ranges that include a peak or a bottom of a light intensity spectrum of light reflected from the fundus, the processing unit 33 may select a wavelength range corresponding to biometric information of a subject to be examined and generate a fundus image corresponding to the selected wavelength range from a compressed image. In the first embodiment, the processing unit 33 determines the user U's blood glucose level.

The storage unit 32 and the processing unit 33 may be separate components, or they may be integrated into a single component such as a microcontroller unit.

### 1-14. User Terminal

The user terminal 40 may be, for example, a terminal owned by the user U. The user terminal 40 may have, for example, a processor, a memory, a communication interface, and a display apparatus. The user terminal 40 may display the results of the determination of the biometric information received from the image processing system 30A on the display apparatus. The user terminal 40 includes, for example, a personal computer, a tablet computer, and a smartphone.

### 1-15. Determination of Biometric Information

A process of determining biometric information will then be described using a process of determining blood glucose levels as an example. Blood glucose levels can be determined from a light absorption spectrum of glucose contained in blood. The light absorption spectrum of glucose has peaks and troughs in the wavelength range of short-wave infrared light. Specifically, peaks or troughs are included in the wavelength range of 920 nm to 980 nm, inclusive, the wavelength range of 1040 nm to 1080 nm, inclusive, and the wavelength range of 1120 nm to 1160 nm, inclusive. Therefore, blood glucose levels can be determined based on the intensities of lights of these wavelength ranges in light intensity spectra of the light absorption spectrum of glucose. The following describes an example of a process of determining blood glucose levels based on the intensity of short-wave infrared light. In the following, the wavelength range of 920 nm to 980 nm, inclusive, may be referred to as the "first determination wavelength range", the wavelength range of 1040 nm to 1080 nm, inclusive, may be referred to as the "second determination wavelength range", and the wavelength range of 1120 nm to 1160 nm, inclusive, may be referred to as the "third determination wavelength range".

The processing unit 33 extracts, from the fundus images for each wavelength range, at least one of a fundus image of a wavelength range corresponding to the first determination wavelength range, a fundus image of a wavelength range corresponding to the second determination wavelength range, and a fundus image of a wavelength range corresponding to the third determination wavelength range. Alternatively, when performing the reconstruction process, the processing unit 33 generates, from a compressed image, at least one of a fundus image of a wavelength range corresponding to the first determination wavelength range, a fundus image of a wavelength range corresponding to the second determination wavelength range, and a fundus image of a wavelength range corresponding to the third determination wavelength range. Below, a process of determining blood glucose levels using the fundus image of the wavelength range corresponding to the first determination wavelength range will be described as an example. Hereinafter, the fundus image of the wavelength range corresponding to the first determination wavelength range will be referred to as the "fundus image to be determined", and the wavelength range corresponding to the first determination wavelength range will be referred to as the "specific wavelength range".

The processing unit 33 obtains the intensity of light in a specific wavelength range included in a light intensity spectrum of light reflected from retinal vessels RV based on images of the retinal vessels RV included in the fundus image to be determined. More specifically, the processing unit 33 identifies images of the retinal vessels RV included in the fundus image to be determined by image processing. For example, the processing unit 33 may identify images of the retinal vessels RV included in the fundus image to be determined by edge detection processing or object detection processing. Once the processing unit 33 has identified images of the retinal vessels RV, the processing unit 33 identifies an arbitrary part within the retinal vessels RV. Then, the processing unit 33 obtains the intensity of light of an image of the arbitrary part identified within the retinal vessels RV. Hereinafter, the intensity of light in a specific wavelength range included in a light intensity spectrum of light reflected from the retinal vessels RV may be referred to as the "light intensity to be determined".

The processing unit 33 calculates the intensity of light in a specific wavelength of a light absorption spectrum based on the intensity of light in the specific wavelength range included in a light intensity spectrum of light emitted from the light source 14 and the light intensity to be determined. Then, the processing unit 33 determines the blood glucose level of the user U based on the intensity of light in the specific wavelength range of the light absorption spectrum. The light intensity spectrum of the light emitted from the light source 14 is stored in advance in the storage unit 32.

The process for determining the user U's blood glucose level using the first determination wavelength range has been described above as an example. However, the processing unit 33 may determine the user U's blood glucose level based on fundus images for wavelength ranges corresponding to the second determination wavelength range or the third determination wavelength range. Alternatively, the processing unit 33 may determine the user U's blood glucose level based on fundus images for wavelength ranges corresponding to two or three of the first, second, and third determination wavelength ranges.

In the first embodiment, the light intensity spectrum of the light emitted from the light source 14 is pre-stored in the storage unit 32. However, at least one of the intensity of light in the first determination wavelength range, the intensity of light in the second determination wavelength range, and the intensity of light in the third determination wavelength range, among the intensities of light included in the light intensity spectrum of the light emitted from the light source 14, may be pre-stored in the storage unit 32.

### 1-16. Compressed Image Data, Hyperspectral Filter, and Reconstruction Process

The compressed image data, a configuration of the hyperspectral filter 221a, and the reconstruction process will then be described.

### 1-16-1. Compressed Image Data

Compressed image data can be obtained, for example, by utilizing known compression sensing techniques. More specifically, the hyperspectral filter 221a has an incident surface into which light from the subject is incident. The hyperspectral filter 221a transmits the light incident on the incident surface with different light transmission characteristics depending on regions of the incident surface. For example, the hyperspectral filter 221a has a plurality of filters respectively corresponding to pixels of the image sensor 222. Each filter has its own unique light transmission characteristics. The hyperspectral filter 221a may be configured such that the light transmission characteristics of each filter are irregularly arranged in the plane direction of the incident surface.

The process through which the hyperspectral filter 221a transmits light with different light transmission characteristics depending on regions of the incident surface is also called "encoding", and the hyperspectral filter 221a is sometimes called an "encoding mask". Through encoding, the intensity of light in each of more than three wavelength ranges included in the target wavelength band λ is modulated.

By performing imaging using the hyperspectral filter 221a, compressed image data is obtained in which pieces of image information from each wavelength range are compressed into a single two-dimensional image data. In the compressed image data, the spectral information of the subject is compressed and recorded as one pixel value for each pixel. In other words, each pixel included in a compressed image contains pieces of information from each wavelength range.

### 1-16-2. Configuration of Hyperspectral Filter

FIG. 5 is a schematic diagram illustrating a configuration of the hyperspectral filter 221a. FIG. 5 illustrates the hyperspectral filter 221a as viewed from the direction of incidence of light entering the hyperspectral filter 221a. As shown in FIG. 5, the hyperspectral filter 221a has a plurality of light-transmitting filters arranged in two dimensions along a plane that intersects the optical path of light incident on the hyperspectral filter 221a. The plurality of filters includes two or more filters whose wavelength dependence of light transmittance differs from each other.

In the example shown in FIG. 5, the hyperspectral filter 221a has 100 filters arranged in a 10×10 grid. FIG. 5 is illustrative, and the number of filters in the hyperspectral filter 221a is not limited to 100. The number of filters in the hyperspectral filter 221a may be the same as or different from the number of pixels in the image sensor 222.

The hyperspectral filter 221a may be constructed using, for example, mirrors, multilayer films, organic materials, diffraction grating structures, etc. Such multilayer films may include, for example, dielectric multilayer films or metal layers. In this case, each filter is formed such that at least one of a thickness and a material of the multilayer film is different. This configuration allows for different spectral characteristics to be achieved depending on the filter.

FIG. 6 shows a relationship between the target wavelength band λ and a plurality of wavelength ranges λ1, λ2, ..., λn included in the target wavelength band λ. Here, "n" is any integer greater than or equal to 4. The target wavelength band λ can be set to various ranges depending on biometric information of a subject to be examined. For example, when determining blood glucose levels, the target wavelength band λ includes the wavelength range of short-wave infrared light from 1000 nm to 1600 nm. In addition, a predetermined number (n) of wavelength ranges can be arbitrarily set relative to the target wavelength band λ. When determining blood glucose levels, "n" can be any integer greater than or equal to 20. For example, "n" may be 30.

The example shown in FIG. 6 illustrates n wavelength ranges λ1, λ2, …, λn obtained by dividing the target wavelength band λ into n equal parts. However, the bandwidth may be non-uniform depending on the wavelength range. There may also be gaps between adjacent wavelength ranges.

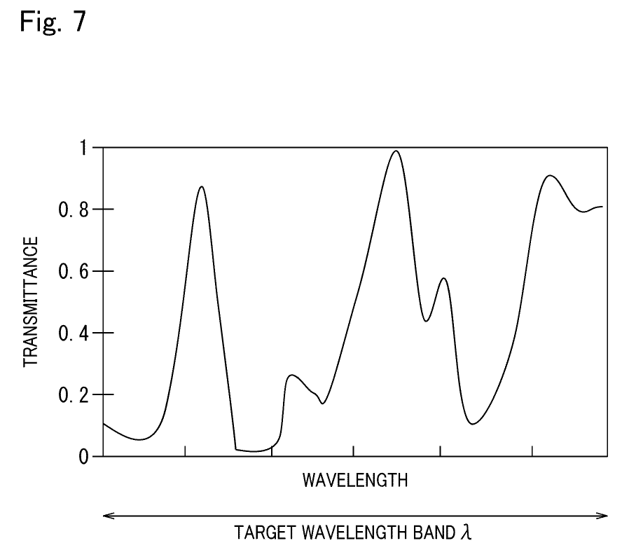

FIG. 7 shows an example of a light transmission spectrum of one filter included in the hyperspectral filter 221a. More specifically, FIG. 7 shows an example of a light transmission spectrum in the target wavelength band λ. In FIG. 7, the horizontal axis represents the wavelength of light, and the vertical axis represents the transmittance of light. As explained with reference to FIG. 6, n wavelength ranges are set for the target wavelength band λ. As shown in FIG. 7, the light transmittance of the filter has a maximum value in two or more wavelength ranges included in the n wavelength ranges.

The hyperspectral filter 221a has two or more filters whose light transmission spectra differ from each other. In one example, the number of light transmission spectrum patterns of the plurality of filters included in the hyperspectral filter 221a may be equal to or greater than the number of wavelength ranges n included in the target wavelength band λ. The hyperspectral filter 221a may be designed so that more than half of the filters have different light transmission spectra, or the hyperspectral filter 221a may be designed so that all of the filters have different light transmission spectra.

FIG. 8 shows an example of the light transmittance of the hyperspectral filter 221a. More specifically, FIG. 8 shows an example of a spatial distribution of light transmittance for a plurality of wavelength ranges λ1, λ2, …, λn included in the target wavelength band λ. In the example shown in FIG. 8, the difference in the shades of black or white of each filter represents the difference in light transmittance. Lighter black areas have higher light transmittance, while darker black areas have lower light transmittance. As shown in FIG. 8, the spatial distribution of light transmittance differs depending on the wavelength range.

Data showing the spatial distribution characteristics of the light transmittance of the hyperspectral filter 221a is acquired in advance based on design data, simulation data, or measured data, and is used to create the reconstruction table 322.

### 1-16-3. Reconstruction Process

The processing unit 33 of the image processing system 30A performs reconstruction process based on the compressed image data received from the wearable device 101 and the reconstruction table 322. The compressed image data includes, for example, image information for wavelength range λ1, image information for wavelength range λ2,..., image information for wavelength range λn. The reconstruction table 322 may be, for example, data showing the spatial distribution characteristics of the light transmittance of the hyperspectral filter 221a. By using the spatial distribution characteristics information of the light transmittance of the hyperspectral filter 221a, images of a plurality of wavelength ranges included in the target wavelength band λ can be generated from a single compressed image. Hereinafter, the image generated from the compressed image may be referred to as a "reconstructed image".

In the reconstruction process, the processing unit 33 may derive the pixel values of all pixels for each wavelength range included in the target wavelength band λ based on the compressed image data and the reconstruction table 322, and generate a reconstructed image for each wavelength range.

Alternatively, the processing unit 33 may perform the following process to estimate at least some of the pixel values, instead of deriving the pixel values of all pixels, or in addition to doing so. That is, in a typical image, adjacent pixels often have colors or pixel values that are somewhat close. Similarly, it has been found that in a reconstructed image, adjacent pixels are connected to a certain extent with respect to color or pixel value. Based on this finding, the processing unit 33 may estimate the reconstructed image in the reconstruction process such that adjacent pixels are connected smoothly with respect to color or pixel value. This makes it possible to reduce a processing load on the processing unit 33 while maintaining the accuracy of reconstruction.

Details of the reconstruction process that generates a plurality of reconstructed images corresponding to respective wavelength ranges included in the target wavelength band λ from a compressed image in which the spectral information of an object is compressed based on the spatial distribution characteristics of the light transmittance for each wavelength range of the hyperspectral filter 221a are disclosed, for example, in WO2024/142683. The entirety of the disclosure in WO2024/142683 is incorporated herein by reference.

### 1-17. Fundus Image

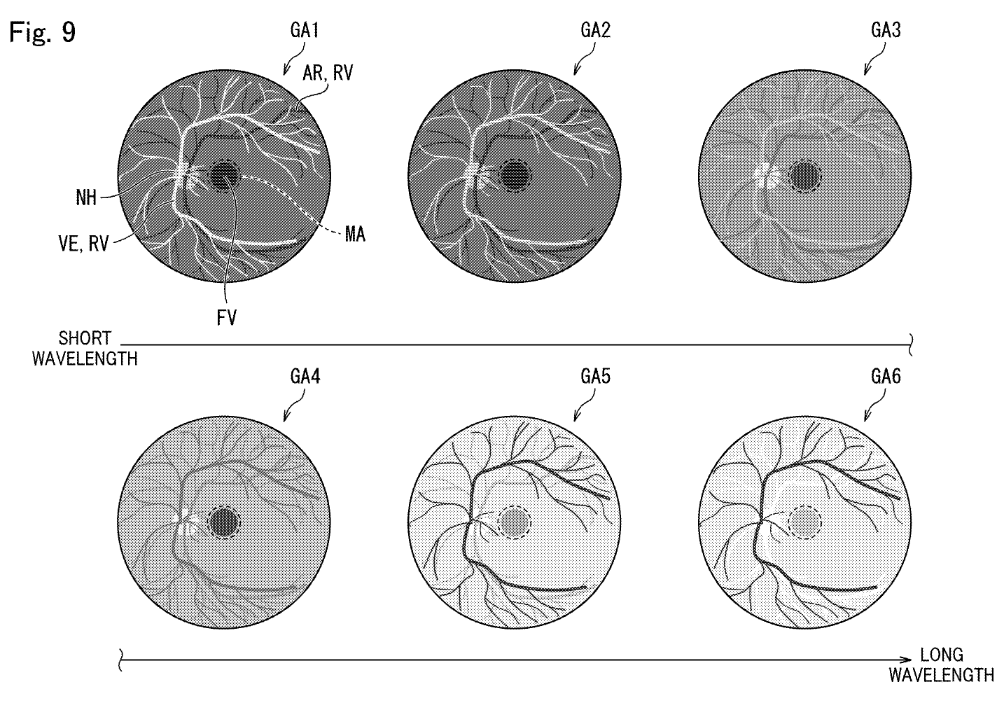

Referring then to FIG. 9, the fundus image for each wavelength range generated from a compressed image will be described. FIG. 9 is a diagram showing an example of fundus images for respective wavelength ranges generated from a compressed image. Specifically, FIG. 9 shows an example of a first fundus image GA1, a second fundus image GA2, a third fundus image GA3, a fourth fundus image GA4, a fifth fundus image GA5, and a sixth fundus image GA6 generated from a single compressed image. The first fundus image GA1, the second fundus image GA2, the third fundus image GA3, the fourth fundus image GA4, the fifth fundus image GA5, and the sixth fundus image GA6 show fundus images consisting of light images from different wavelength ranges.

As shown in FIG. 9, the fundus includes a fovea FV, a macula MA, an optic nerve head NH, an arterial vessel AR, and a venous vessel VE. The arterial vessel AR and venous vessel VE are retinal vessels RV. The fovea FV is located in the center of the fundus. The macula MA is the peripheral part of the fovea FV. The optic nerve head NH is observed to be brighter than other parts of the fundus. The retinal vessels RV extend from the optic nerve head NH.

As shown in FIG. 9, the intensity of light reflected from the fundus differs depending on the wavelength range. Accordingly, by obtaining the intensity of light from fundus images for respective wavelength ranges, it is possible to obtain a light intensity spectrum of light reflected from the fundus.

As already described, when determining blood glucose levels, the processing unit 33 identifies an arbitrary site within the retinal vessels RV. As shown in FIG. 9, each retinal vessel RV includes a thick vessel extending from the optic nerve head NH and a plurality of thin vessels branching off from the thick vessel. The thin vessels become thinner as they approach their ends. Therefore, by identifying a thick part within the retinal vessels RV as an arbitrary site within the retinal vessels RV, the processing unit 33 can acquire a light intensity spectrum of light reflected from the retinal vessels RV with higher accuracy.

Furthermore, as described referring to FIG. 4, the image-forming optical system 21 may include a zoom lens. In a configuration in which the image-forming optical system 21 includes a zoom lens, the light intensity spectrum of the light reflected from the retinal blood vessels RV can be obtained with even higher accuracy by magnifying and imaging the thick part of the retinal blood vessels RV.

### 2. Operation

### 2-1. Correction Process for Imaging Position and Focusing Process

Next, an operation of the imaging apparatus 10A will be described with reference to FIG. 10. FIG. 10 is a flowchart showing a flow of processing performed by the controller 17 included in the imaging apparatus 10A before capturing images of the fundus of the user U. The processing shown in FIG. 10 is started, for example, when the user U attaches the wearable device 101 to the user U's head and operates the operating section 15.

Specifically, the process shown in FIG. 10 includes a process for correcting an image position and a focusing process. The operating section 15 further receives an operation to command the start of the process for correcting an image position and an operation to command the start of the focusing process. The operation to command the start of the process for correcting an image position and the operation to command the start of the focusing process may be one operation or they may be different operations. The process shown in FIG. 10 shows the process that is executed in a configuration in which the operation to command the start of the process for correcting an image position and the operation to command the start of the focusing process are one operation.

In detail, when the user U determines biometric information from the fundus image of the right eye E1, the user U operates the operating section 15 to set an imaging mode of the imaging apparatus 10A to a mode for imaging the fundus of the right eye E1. After that, the user U operates the operating section 15 to cause the controller 17 to perform the process for correcting an imaging position and the focusing process. The user U operates the operating section 15 in the same way when determining biometric information from the fundus image of the left eye E2. Hereinafter, the process shown in FIG. 10 will be described using the right eye E1 as an example.

The process shown in FIG. 10 includes steps S1, S2, S3, S4, and S5. When the controller 17 starts the process shown in FIG. 10, the controller 17 controls the imaging device 22 to start imaging of the user U's right eye E1. As a result, compressed image data is generated (step S1).

The controller 17 detects an amount of displacement of the imaging position based on a compressed image data (step S2). Here, the imaging position indicates a position of the fundus within a compressed image (two-dimensional image). More specifically, the controller 17 detects the amount of displacement and a direction of the displacement. The amount of displacement indicates an amount of displacement between the position of the fundus within the compressed image and a first predetermined position. The direction of the displacement indicates a direction in which the position of the fundus is displaced from the first predetermined position. For example, the controller 17 identifies a position of a fovea FV included in the compressed image and calculates an amount of displacement between the position of the fovea FV and a second predetermined position as the amount of displacement of the imaging position. The controller 17 also calculates a direction in which the fovea FV is displaced from the second predetermined position. Alternatively, the controller 17 may calculate the amount of displacement and the direction of the displacement based on a position of the eyelid or pupil of the user included in the compressed image.

The controller 17 controls the sensor driving unit 25 based on the calculated amount of displacement to move the imaging device 22, thereby reducing the amount of displacement or making it zero. As a result, the displacement of the imaging position is corrected. Specifically, the controller 17 controls the sensor driving unit 25 based on the calculated amount of the displacement and the calculated direction of the displacement to move the imaging device 22.

Specifically, the controller 17 calculates a correction amount to correct the displacement of the imaging position based on the calculated amount of displacement (step S3). For example, the controller 17 calculates a correction amount to match the position of the fovea FV to the second predetermined position. Here, the correction amount represents an amount of movement of the imaging device 22. Furthermore, the controller 17 calculates a direction in which to move the imaging device 22 based on the calculated direction of the displacement. More specifically, the controller 17 calculates the amount of movement of the imaging device 22 and the direction in which to move the imaging device 22 based on the calculated correction amount, the calculated direction of the displacement, and a current position of the imaging device 22 detected by the position sensor 24.

The controller 17 drives the sensor driving unit 25 based on the calculated amount of movement of the imaging device 22 and the calculated direction in which to move the imaging device 22. As a result, the deviation of the imaging position is corrected (step S4).

After correcting the displacement of the imaging position, the controller 17 performs a focusing process to focus on the fundus image (step S5). As a result, the process shown in FIG. 10 is completed. For example, the controller 17 controls a focus position of the focusing lens 211 by moving at least one of the one or more lenses of the focusing lens 211 via the focusing lens driving unit 23 so that a contrast value of the compressed image is maximized. Alternatively, the controller 17 may control the focus position of the focusing lens 211 by an edge detection method. For example, the controller 17 may control the focus position of the focusing lens 211 by determining whether or not edges of retinal blood vessels RV are detected from the compressed image.

As described above with reference to FIG. 10, according to the first embodiment, the imaging position is corrected. Furthermore, the fundus image is focused. For example, since head sizes differ from person to person, the imaging position may be displaced from the first predetermined position depending on the size of the head of the person wearing the wearable device 101. Also, the fundus image may not be in focus. Alternatively, a posture of the wearable device 101 attached to the head may differ from person to person. For this reason, the imaging position may be displaced from the first predetermined position, or the fundus image may not be in focus, depending on the person using the wearable device 101. In contrast, according to the first embodiment, the imaging position is corrected by the process for correcting the imaging position. Furthermore, the fundus image is focused by the focusing process. Therefore, the fundus of the user U is imaged with greater accuracy.

Furthermore, according to the first embodiment, since the light source 14 functions as a guide light source, the light source 14 directs the line of sight of the user U's eye E toward the imaging unit 20 when the process for correcting the imaging position is executed. Therefore, the imaging position is corrected with greater precision. Similarly, the light source 14 directs the line of sight of the user U's eye E toward the imaging unit 20 when the focusing process is executed. Therefore, the focus is adjusted to the fundus image with greater precision.

In the first embodiment, the area around the user U's eye E is shielded from light by the light-shielding portion 11. Therefore, an exposure time need not be adjusted. In other words, the exposure time may be a fixed value. More specifically, the exposure time may be set in advance according to the type of light source 14.

### 2-2. Examining Method

Next, an examining method of the first embodiment will be described with reference to FIG. 11. FIG. 11 is a flowchart showing a flow of the examining method according to the first embodiment of the present disclosure. The examining method of the first embodiment is performed by the imaging apparatus 10A of the wearable device 101 and the image processing system 30A. The examining method shown in FIG. 11 includes steps S11, S12, S13, S14 and S15.

In the following, the process shown in FIG. 11 will be described using the case where the blood glucose level of the user U is determined based on the fundus image of the right eye E1 as an example. The process shown in FIG. 11 is started, for example, when the user U operates the operating section 15 to instruct the start of imaging after the process for correcting the imaging position and the focusing process for the right eye E1 have been performed.

As shown in FIG. 11, when the user U operates the operating section 15 to instruct the start of imaging, the controller 17 of the imaging apparatus 10A controls the imaging device 22 to image the user U's right eye E1. As a result, compressed image data showing the fundus image of the right eye E1 is generated (step S11). The compressed image data is a two-dimensional image data in which multiple pieces of fundus image information for respective wavelength ranges included in the target wavelength band λ are superimposed. In a situation in which the biometric information to be examined is blood glucose level, the target wavelength band λ includes the wavelength range of short-wave infrared light.

The controller 17 controls the communication interface 16 to send the compressed image data to the image processing system 30A (step S12).

When the processing unit 33 of the image processing system 30A receives compressed image data from the wearable device 101 via the communication interface 31, the processing unit 33 performs a reconstruction process based on the examining program 321 (step S13). Specifically, the processing unit 33 generates fundus images for respective wavelength ranges included in the target wavelength band λ based on the compressed image data and the reconstruction table 322.

When the processing unit 33 generates fundus images for respective wavelength ranges, the processing unit 33 executes a determination process based on the examining program 321 (step S14). Specifically, the processing unit 33 determines the user U's biometric information based on the fundus images for respective wavelength ranges. In this case, the processing unit 33 determines the user U's blood glucose level based on the fundus images for respective wavelength ranges.

When the processing unit 33 determines the user U's biometric information, the processing unit 33 executes a notification process based on the examining program 321 (step S15). Specifically, the processing unit 33 transmits a determination result of the biometric information to the user terminal 40 via the communication interface 31. Here, the result of determining the user U's blood glucose level is notified to the user terminal 40. As a result, the examining method shown in FIG. 11 is completed.

The image processing system 30A may further include an operating section and a display apparatus. The operating section is a user interface that receives operations from an operator. When the operating section receives an operation from an operator, the operating section transmits an operation signal corresponding to the operation from the operator to the processing unit 33. In a configuration in which the image processing system 30A further includes an operating section and a display apparatus, the controller 17 of the imaging apparatus 10A may control the imaging device 22 to take a plurality of consecutive images of the fundus of the user U's eye E, and control the communication interface 16 to transmit a plurality of pieces of compressed image data to the image processing system 30A. For example, since the imaging duration for one image taken with a hyperspectral camera is several tens of milliseconds, it is possible to take 10 to 100 images within one second.

When the communication interface 31 receives a plurality of pieces of compressed image data, the processing unit 33 of the image processing system 30A may display a plurality of compressed images (two-dimensional images) on a display apparatus and allow the person in charge to select a compressed image in good condition from among the plurality of compressed images. As a result, the person in charge can operate the operating section to cause the processing unit 33 to perform a reconstruction process to generate fundus images for respective wavelength ranges from the selected compressed image data. Therefore, biometric information is determined with higher accuracy.

Furthermore, since a computational load of the reconstruction process is large, if the reconstruction process is performed on all compressed image data, a processing load on the processing unit 33 may become heavy. In contrast, by configuring the system to perform the reconstruction process only on the selected compressed image data, the processing load on the processing unit 33 is reduced.

The person in charge is a medical professional. However, if the act of "selecting a compressed image in good condition from among compressed images" does not constitute a medical act, the person in charge may include someone other than a medical professional. For example, the user U may be included in the person in charge.

The first embodiment of this disclosure has been described above with reference to FIGS. 1-11. In the first embodiment, the biometric information to be examined is blood glucose level, but the biometric information to be examined is not limited to blood glucose level. The biometric information to be examined can be any biometric information that can be examined from a fundus image. For example, in addition to blood glucose level, the biometric information to be examined may include blood oxygen saturation, retinopathy, and Alzheimer's disease.

### 3. Effects

According to the first embodiment, a subject can use the wearable device 101 to examine their own biometric information.

According to the first embodiment, a subject's biometric information is examined non-invasively. Thus, compared to, for example, the case of collecting a blood sample and examining biometric information based on the blood sample, a physical burden on the subject can be reduced.

According to the first embodiment, the imaging apparatus 10A simultaneously captures light components in a plurality of wavelength ranges contained in light reflected from the fundus of the subject's eye, thereby generating image information used to determine the subject's biometric information. Therefore, compared to configurations that require processing to scan laser light or processing to sweep the wavelength of laser light, a time required to image the fundus of the subject is shortened. For example, when determining blood glucose levels, the imaging apparatus 10A captures light in the wavelength range of 920 nm to 980 nm, inclusive, light in the wavelength range of 1040 nm to 1080 nm, inclusive, and light in the wavelength range of 1120 nm to 1160 nm, inclusive, in a single shot. Therefore, a time required to image the fundus of the subject is shortened. For example, a time required for a single image taken with a hyperspectral camera is several tens of milliseconds.

According to the first embodiment, a time required to image the subject's fundus is shortened, thus reducing a time the subject has to open their eyelids and expose their eyeballs. Accordingly, a physical burden on the subject can be reduced. In addition, since a time the subject has to maintain an open eyelid state is shortened, the fundus image is less likely to blur. Thus, a more stable fundus image can be acquired, and the subject's biometric information can be determined with higher accuracy.

Furthermore, when using a configuration that scans with the laser beam, there is a risk that the subject's pupils may constrict during the scan. If the subject's pupils constrict during the scan, the process of scanning the laser beam must be repeated. Similarly, when using a configuration that sweeps the wavelength of the laser beam, there is a risk that the subject's pupils may constrict during the sweeping of the wavelength of the laser beam. If the subject's pupils constrict during the sweeping of the wavelength of the laser beam, the process of sweeping the wavelength of the laser beam must be repeated. In contrast, according to the first embodiment, a time required to image the subject's fundus is shortened, thus reducing the risk of the subject's pupils constricting during imaging. Therefore, it is possible to reduce the probability that the imaging process needs to be repeated.

According to the first embodiment, a time required to image the subject's fundus is shortened, making it possible to perform the examination without using a mydriatic agent. Therefore, a physical burden on the subject can be reduced.

According to the first embodiment, light in each wavelength range included in the target wavelength band λ is simultaneously captured in each pixel of the image sensor 222. Therefore, the sensitivity of the imaging device 22 is increased. Thus, the illuminance of the light source 14 can be set to a low value such that the user U's pupils do not constrict. As a result, a physical burden on the subject can be reduced. In addition, by emitting light including visible light from the light source 14, the light source 14 functions as a guide light source.

According to the first embodiment, each pixel of the image sensor 222 simultaneously captures light in each wavelength range included in the target wavelength band λ. Therefore, the spatial resolution of the captured fundus image is high. Thus, the subject's biometric information is determined with greater accuracy.

According to the first embodiment, since a laser oscillator is not used, the imaging apparatus 10A is miniaturized. Also, compared to configurations that require a mechanism for scanning laser light or a mechanism for sweeping the wavelength of laser light, the imaging apparatus 10A is made smaller. Therefore, it becomes easy to mount the imaging apparatus 10A on the wearable device 101.

According to the first embodiment, since the wearable device 101 has the light-shielding portion 11, ingress of ambient light into the imaging unit 20 is suppressed. Therefore, the influence of ambient light is avoided, and the subject's biometric information is determined with higher accuracy.

In the first embodiment, the image processing system 30A is configured as a single apparatus, but the image processing system 30A may be configured by a plurality of apparatuses connected communicably to each other. For example, the image processing system 30A may be configured by an apparatus that performs reconstruction process and an apparatus that performs determination process.

### 4. Variants

### First Variant

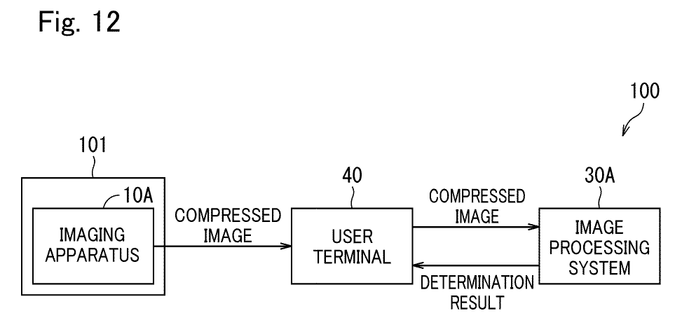

Referring then to FIG. 12, a first variant of the examining system 100 will be described. However, matters different from the matters described in the first embodiment will be described, and descriptions of the same matters as in the first embodiment will be omitted as appropriate.

FIG. 12 shows the examining system 100 according to the first variant. In the examining system 100 described with reference to FIGS. 1-11, compressed image data is transmitted from the imaging apparatus 10A to the image processing system 30A. However, as shown in FIG. 12, the compressed image data may also be transmitted from the imaging apparatus 10A to the image processing system 30A via the user terminal 40. In this case, the user terminal 40 may include application software for transmitting the compressed image data received from the imaging apparatus 10A to the image processing system 30A.

### Second Variant

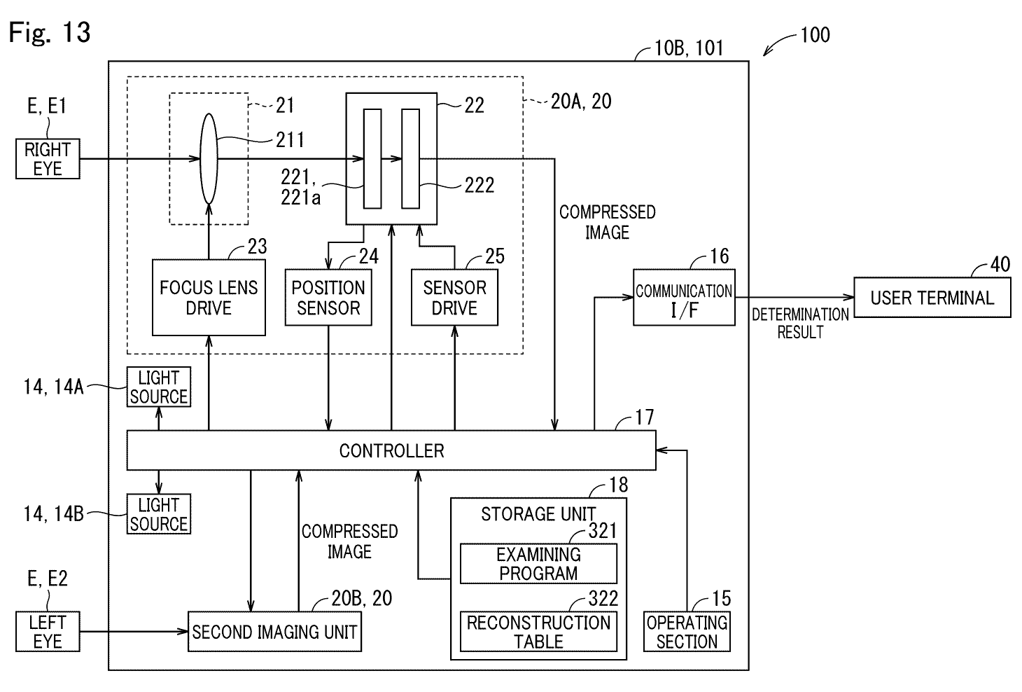

Next, a second variant of the examining system 100 will be described with reference to FIG. 13. However, matters different from the matters described in the first embodiment and the first variant will be described, and descriptions of the same matters as in the first embodiment and the first variant will be omitted as appropriate.

FIG. 13 shows the examining system 100 according to the second variant. In the second variant, the image processing system 30A is omitted from the examining system 100. In other words, in the second variant, the functions of the image processing system 30A are implemented in the wearable device 101, and the examining system 100 is composed solely of the wearable device 101. In the second variant, the wearable device 101 transmits the results of the determination of the biometric information to the user terminal 40.

More specifically, in the second variant, the wearable device 101 has an imaging apparatus 10B. The imaging apparatus 10B includes the light source 14, the operating section 15, the communication interface 16, the controller 17, the first imaging unit 20A, the second imaging unit 20B, and a storage unit 18.

The storage unit 18 is, for example, a semiconductor memory. The storage unit 18 stores the examining program 321 and the reconstruction table 322. The controller 17 of the imaging apparatus 10B, similar to the processing unit 33 of the image processing system 30A, performs reconstruction process to generate reconstructed images for each wavelength range included in the target wavelength band λ. Furthermore, the controller 17 of the imaging apparatus 10B, similar to the processing unit 33 of the image processing system 30A, performs determination process to determine the biometric information of the user U.

After executing the determination process, the controller 17 of the imaging apparatus 10B controls the communication interface 16 to transmit the determination result of the user U's biometric information to the user terminal 40.

Although the imaging apparatus 10B shown in FIG. 13 includes the storage unit 18, the storage unit 18 may be omitted. In this case, the examining program 321 and the reconstruction table 322 may be stored in a memory built into the controller 17.

### Third Variant

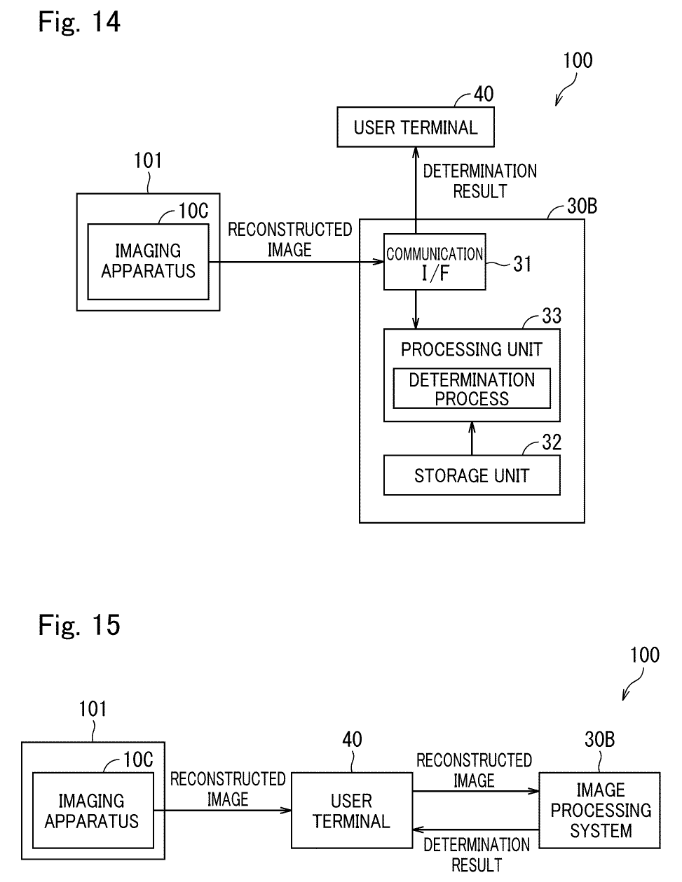

Next, a third variant of the examining system 100 will be described with reference to FIG. 14. However, matters different from the matters described in the first embodiment, the first variant, and the second variant will be described, and descriptions of the same matters as in the first embodiment, the first variant, and the second variant will be omitted as appropriate.

FIG. 14 shows the examining system 100 according to the third variant. In the third variant, the wearable device 101 transmits reconstructed image data to an image processing system 30B.

More specifically, in the third variant, the wearable device 101 includes an imaging apparatus 10C. The controller 17 of the imaging apparatus 10C performs a reconstruction process, similar to the second variant, to generate reconstructed images corresponding to respective wavelength ranges included in the target wavelength band λ. However, unlike the second variant, in the third variant, the controller 17 of the imaging apparatus 10C does not perform the determination process.

After performing the reconstruction process, the controller 17 of the imaging apparatus 10C controls the communication interface 16 to transmit the reconstructed image data to the image processing system 30B.

In the third variant, the image processing system 30B receives reconstructed image data from the wearable device 101. The processing unit 33 of the image processing system 30B performs a determination process using the reconstructed image data received from the wearable device 101 to determine the biometric information of the user U.

### Fourth Variant

Next, a fourth variant of the examining system 100 will be described with reference to FIG. 15. However, matters different from the matters described in the first embodiment and the first to third variants will be described, and descriptions of the same matters as in the first embodiment and the first to third variants will be omitted as appropriate.

FIG. 15 shows the examining system 100 according to the fourth variant. In the third variant described with reference to FIG. 14, the reconstructed image data is transmitted from the imaging apparatus 10C to the image processing system 30B. However, as shown in FIG. 15, the reconstructed image data may be transmitted from the imaging apparatus 10C to the image processing system 30B via the user terminal 40. In this case, the user terminal 40 may include application software for transmitting the reconstructed image data received from the imaging apparatus 10C to the image processing system 30B.

### Fifth Variant

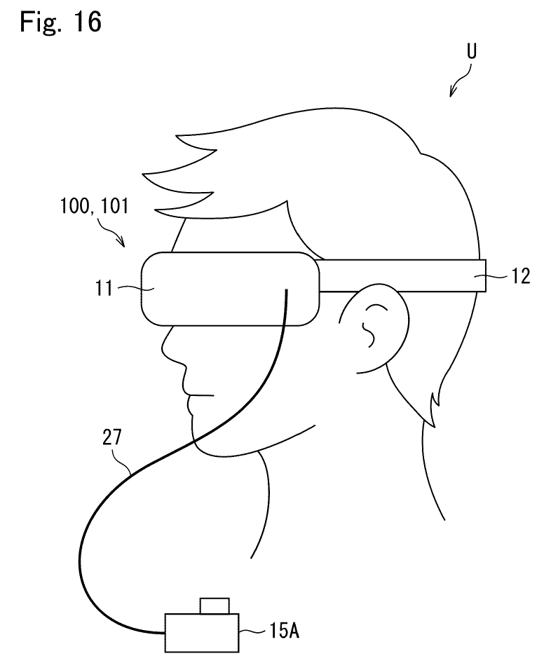

Next, a fifth variant of the examining system 100 will be described with reference to FIG. 16. However, matters different from the matters described in the first embodiment and the first to fourth variants will be described, and descriptions of the same matters as in the first embodiment and the first to fourth variants will be omitted as appropriate.

FIG. 16 shows the examining system 100 according to the fifth variant. In the examining system 100 described with reference to FIG. 1, the operating section 15 is provided in the light-shielding portion 11, but as shown in FIG. 16, the examining system 100 of the fifth variant includes an operating unit 15A and a cable 27 instead of the operating section 15.

The operating unit 15A is located outside the wearable device 101. Like the operating section 15, the operating unit 15A is a user interface that accepts operations from the user U. The cable 27 is a signal line that transmits operation signals from the operating unit 15A to the controller 17 shown in FIG. 4. When the operating unit 15A receives an operation from the user U, the operating unit 15A transmits an operation signal corresponding to the operation to the controller 17 via the cable 27.

The embodiment of the present disclosure has been described above with reference to the drawings (FIGS. 1-16). For example, according to one embodiment of the present disclosure, the examining system 100 examines biometric information of a subject. The examining system 100 includes the imaging unit 20 and the processing unit 33. The imaging unit 20 includes the image sensor 222. The image sensor 222 simultaneously captures light components in more than three wavelength ranges contained in light reflected from the fundus of the subject to generate image information used to determine the biometric information of the subject. The processing unit 33 functions as an image processing circuit and generates an image of the fundus for each wavelength range from the image information. Furthermore, the processing unit 33 functions as a determination processing circuit and determines the biometric information of the subject based on the image of the fundus for each wavelength range. Therefore, since light components in more than three wavelength ranges contained in the light reflected from the fundus of the subject are captured simultaneously, the subject's biometric information can be examined in a relatively short imaging duration.

Furthermore, according to one embodiment of this disclosure, the processing unit 33, which functions as a determination processing circuit, acquires a light intensity spectrum of light reflected from retinal blood vessels RV included in the fundus based on the image of the fundus for each wavelength range, and determines the biometric information of subject based on the acquired light intensity spectrum. Therefore, the subject's biometric information can be determined based on the intensity of light reflected from retinal blood vessels RV. Thus, the subject's biometric information can be determined with greater accuracy.

Furthermore, according to one embodiment of the present disclosure, the image sensor 222 has a plurality of pixels, outputs a signal in which pieces of information from each wavelength range are superimposed from each of the plurality of pixels, and generates the image information using the signals output from each of the plurality of pixels. Therefore, the sensitivity for capturing fundus images can be increased.

Furthermore, according to one embodiment of the present disclosure, the imaging unit 20 further includes the hyperspectral filter 221a. The hyperspectral filter 221a is an example of a filter array positioned in the optical path of light incident on the image sensor 222. The hyperspectral filter 221a has a plurality of light-transmitting filters arranged two-dimensionally along a plane intersecting the optical path. The plurality of filters includes two or more filters whose wavelength dependence of light transmittance differs from each other. The light transmittance of each of the two or more filters has a maximum value in two or more wavelength ranges included in the more than three wavelength ranges. Therefore, the spatial resolution of the fundus image can be enhanced.

Furthermore, according to one embodiment of the present disclosure, the imaging unit 20 further includes the imaging device 22 having the hyperspectral filter 221a. The examining system 100 further comprises the sensor driving unit 25 that moves the imaging device 22. The controller 17 functions as a control circuit and controls the sensor driving unit 25. Specifically, the controller 17, which functions as a control circuit, calculates an amount of displacement between a position of the fundus in an image indicated by the image information and a predetermined position, and controls the sensor driving unit 25 to move the imaging device 22 based on the amount of displacement so as to reduce the amount of displacement. As a result, when the position of the fundus in the image is displaced from the predetermined position, the position of the fundus in the image is corrected. Accordingly, the fundus of the subject can be imaged with higher accuracy.

Furthermore, according to one embodiment of this disclosure, the examining system 100 further includes the light-shielding portion 11 that shields an area around at least one eye E of the subject. Therefore, visible light is less likely to be irradiated onto the subject's eye E from the surrounding environment. As a result, the subject is less likely to close their eyelids. Consequently, the fundus of the subject's eye can be imaged more reliably.

Furthermore, according to one embodiment of the present disclosure, the light-shielding portion 11 has the front wall portion 11a facing the front of the head of the subject and the support portion 13 that supports the front wall portion 11a. The imaging unit 20 is supported by the front wall portion 11a, and the support portion 13 maintains a predetermined distance between at least one eye E of the subject and the imaging unit 20. Therefore, focusing process to focus on the fundus of the eye E becomes easier.

Furthermore, according to one embodiment of the present disclosure, the examining system 100 further comprises at least one of the first light source 14A that emits light to illuminate the subject's right eye E1 and the second light source 14B that emits light to illuminate the subject's left eye E2, and at least one of the diffuser 26A that diffuse the light emitted from the first light source 14A and the diffuser 26B that diffuse the light emitted from the second light source 14B. By disposing the diffuser 26A, the fundus of the subject's right eye E1 can be illuminated uniformly. As a result, the fundus of the subject's right eye E1 can be imaged more reliably. Similarly, by disposing the diffuser 26B, the fundus of the subject's left eye E2 can be illuminated uniformly. As a result, the fundus of the subject's left eye E2 can be imaged more reliably.

Furthermore, according to one embodiment of the present disclosure, the examining system 100 further includes a guide light source that emits guide light to direct the line of sight of the subject toward the imaging unit 20. For example, the examining system 100 can make the light source 14 function as a guide light source. Therefore, since the line of sight of the subject can be directed toward the imaging unit 20, the fundus of the subject's eye can be imaged more reliably.

Furthermore, according to one embodiment of the present disclosure, the examining system 100 further comprises the wearable device 101 that is attached to the head of the subject, and the wearable device 101 has the imaging unit 20. Therefore, the subject can have their own biometric information examined by attaching the wearable device 101 to their head.

Furthermore, according to one embodiment of this disclosure, the light incident on the imaging unit 20 includes infrared light. Therefore, the subject's biometric information can be examined using infrared light.

Furthermore, according to one embodiment of this disclosure, the biometric information includes blood glucose levels. Therefore, blood glucose levels can be determined.

Furthermore, according to one embodiment of the present disclosure, the image processing system 30A examines biometric information of a subject. The image processing system 30A comprises the communication interface 31 and the processing unit 33. The communication interface 31 acquires image information from the imaging unit 20. The imaging unit 20 simultaneously captures light components in more than three wavelength ranges contained in light reflected from the fundus of the subject to generate the image information used to determine the biometric information of the subject. The processing unit 33 functions as an image processing circuit and generates an image of the fundus for each wavelength range from the image information. Furthermore, the processing unit 33 functions as a determination processing circuit and determines the biometric information of the subject based on the image of the fundus for each wavelength range. Therefore, by using the image processing system 30A to examine the subject's biometric information, the imaging unit 20 can be used to capture the subject's fundus. As a result, the subject's biometric information can be examined in a relatively short imaging duration.

Furthermore, according to one embodiment of the present disclosure, a non-transitory computer-readable storage medium is a non-transitory computer-readable storage medium storing the examining program 321. The examining program 321 is a computer program for examining biometric information of a subject. The examining program 321 causes a computer to execute processes including: acquiring image information used to determine the biometric information of the subject from the imaging unit 20 that simultaneously captures light components in more than three wavelength ranges contained in light reflected from the fundus of the subject to generate the image information; generating an image of the fundus for each wavelength range from the image information; and determining the biometric information of the subject based on the image of the fundus for each wavelength range. Therefore, by using the storage medium storing the examining program 321 to examine the subject's biometric information, the imaging unit 20 can be used to capture the subject's fundus. As a result, the subject's biometric information can be examined in a relatively short imaging duration.

Furthermore, according to one embodiment of the present disclosure, the wearable device 101 is attached to the head of the subject. The wearable device 101 comprises the imaging unit 20 and the light-shielding portion 11. The imaging unit 20 includes the image sensor 222. The image sensor 222 simultaneously captures light components in more than three wavelength ranges contained in light reflected from the fundus of the subject to generate image information used to determine the biometric information of the subject. The light-shielding portion 11 shields an area around at least one eye E of the subject from light. Therefore, since light components in more than three wavelength ranges contained in the light reflected from the subject's fundus are captured simultaneously, the subject's biometric information can be examined in a relatively short imaging duration. In addition, visible light from the subject's surrounding environment is less likely to irradiate the subject's eye E, making it easier for the subject's pupil to dilate. Thus, the fundus of the subject can be captured more reliably.

Furthermore, according to one embodiment of the present disclosure, the examining method is a method for examining biometric information of a subject. The examining method includes: imaging the fundus of the subject with the image sensor 222 to generate image information used to determine the biometric information of the subject, the image sensor 222 simultaneously capturing light components in more than three wavelength ranges contained in the incident light; generating an image of the fundus for each wavelength range from the image information with the processing unit 33 that functions as an image processing circuit; and determining the biometric information of the subject based on the image of the fundus for each wavelength range with the processing unit 33 that functions as a determination processing circuit. Therefore, since light components in more than three wavelength ranges contained in the light reflected from the fundus of the subject are captured simultaneously, the subject's biometric information can be examined in a relatively short imaging duration.

The present disclosure is not limited to the embodiment described above, and can be carried out in various forms without departing from its gist. Furthermore, the components disclosed in the embodiment can be modified as appropriate. For example, some of all components shown in one embodiment may be added to the components of another embodiment, or some of all components shown in one embodiment may be removed from the embodiment.

The drawings schematically show each component in order to facilitate understanding, and the thickness, length, number, spacing, etc. of each component shown may differ from the actual dimensions due to the constraints in creating the drawings. Furthermore, the configuration of each component shown in the above embodiment is merely an example and is not particularly limitative, and naturally, various modifications are possible within the scope that does not substantially deviate from the effects of this disclosure.

For example, in the embodiment described with reference to FIGS. 1-16, the filter unit 221 has a hyperspectral filter 221a, but the filter unit 221 only needs to have an optical element that simultaneously directs, to the image sensor 222, the light components in more than three wavelength ranges included in the target wavelength band λ. For example, the filter unit 221 may have a multispectral filter. In other words, the imaging apparatus 10A, 10B and 10C may be multispectral cameras. Alternatively, the filter unit 221 may have an optical element such as a prism or grating that spectrally separates the incident light into light beams in more than three wavelength ranges.

In the embodiment described with reference to FIGS. 1-16, the light emitted from the first light source 14A is irradiated directly or via the diffuser plate 26A onto the user U's eye E. However, the light emitted from the first light source 14A may also be irradiated onto the user U's right eye E1 via a half mirror that reflects visible light and transmits infrared light. In this case, the half mirror may be placed, for example, between the user U's right eye E1 and the image-forming optical system 21. The light emitted from the second light source 14B may also be irradiated onto the user U's left eye E2 via a half mirror, similar to the light emitted from the first light source 14A.

In the embodiment described with reference to FIGS. 1-16, the image sensor 222 has a plurality of pixels, and light components in a plurality of wavelength ranges are incident on the pixels of the image sensor 222 simultaneously. However, the image sensor 222 may also have a plurality of main pixels, and light components in a plurality of wavelength ranges may be incident on the main pixels simultaneously. Here, one main pixel is one image consisting of a plurality of subpixels. For example, light beams in different wavelength ranges may be incident on the subpixels included in the main pixel.

In the embodiment described with reference to FIGS. 1-16, the wearable device 101 is of the goggle type, but the form of the wearable device 101 is not particularly limited as long as it can be attached to the head of the user U. For example, the wearable device 101 may be of the helmet type.

In the embodiment described with reference to FIGS. 1-16, the light source 14 is used as a guide light source, but a separate guide light source may be provided.

In the embodiment described with reference to FIGS. 1-16, the wearable device 101 has a halogen lamp or a visible light LED as a guide light source, but the guide light source is not limited to the halogen lamp and visible light LED. The guide light source may also be a display apparatus that displays an object. The display apparatus may be, for example, an LED display. Specifically, a half-mirror that reflects visible light and transmits infrared light may be placed in the optical path through which light reflected from the fundus of the eye enters the image-forming optical system 21, and the visible light emitted from the display apparatus may be irradiated to the user U's eye E via the half-mirror. The object may be, for example, a balloon or an airplane.

In the embodiment described with reference to FIGS. 1-16, the wearable device 101 has the communication interface 16 as an output unit, but the wearable device 101 may also have a display apparatus for displaying the determination result as an output unit. The display apparatus may be, for example, a liquid crystal display apparatus or an LED display.

In the embodiment described with reference to FIGS. 1-16, the imaging apparatus 10A, 10B, and 10C are mounted on the wearable device 101, but the imaging apparatus 10A, 10B, and 10C may also be mounted on a stationary fundus examining apparatus. In this case, at least one of an image processing circuit and a determination processing circuit may be further implemented on the stationary fundus examining apparatus.

In the embodiment described with reference to FIGS. 1-16, autofocus functions is implemented in the imaging apparatus 10A, 10B, and 10C, but the imaging apparatus 10A, 10B, and 10C may also have a focus mechanism that allows the user U to manually adjust the focus.

In the embodiment described with reference to FIGS. 1-16, the imaging unit 20 includes the focusing lens 211 and the focusing lens driving unit 23, but the focusing lens 211 and the focusing lens driving unit 23 may be omitted.

In the embodiment described with reference to FIGS. 1-16, a configuration for correcting the displacement in the imaging position by moving the imaging device 22 is implemented in the imaging apparatus 10A, 10B, and 10C. However, a configuration for correcting the displacement in the imaging position by moving the image-forming optical system 21 may also be implemented in the imaging apparatus 10A, 10B, and 10C. Alternatively, a configuration for correcting the displacement in the imaging position by moving both the imaging device 22 and the image-forming optical system 21 may also be implemented in the imaging apparatus 10A, 10B, and 10C.

In the embodiment described with reference to FIGS. 1-16, the configuration for correcting the displacement in the imaging position is implemented in the imaging apparatus 10A, 10B, and 10C, but the configuration for correcting the displacement in the imaging position may be omitted.

### Notes

The present disclosure further discloses the following aspects, which are not intended to limit this disclosure.

### Aspect 1

An examining system for examining biometric information of a subject, comprising:
an imaging unit including an image sensor, the imaging unit simultaneously capturing, by the image sensor, light components in more than three wavelength ranges contained in light reflected from a fundus of the subject to generate image information used to determine the biometric information of the subject;
an image processing circuit that generates an image of the fundus for each wavelength range from the image information; and
a determination processing circuit that determines the biometric information of the subject based on the image of the fundus for each wavelength range.

### Aspect 2

The examining system according to aspect 1, wherein
the determination processing circuit acquires a light intensity spectrum of light reflected from retinal blood vessels contained in the fundus based on the image of the fundus for each wavelength range, and determines the biometric information of the subject based on the light intensity spectrum acquired.

### Aspect3

The examining system according to aspect 1 or 2, wherein
the image sensor has a plurality of pixels, outputs a signal in which pieces of information from each wavelength range are superimposed from each of the plurality of pixels, and generates the image information using the signal output from each of the plurality of pixels.

### Aspect 4

The examining system according to any one of aspects 1 to 3, wherein
the imaging unit further includes a filter array positioned in an optical path of light incident on the image sensor,
the filter array has a plurality of light-transmitting filters arranged two-dimensionally along a plane intersecting the optical path,
the plurality of filters includes two or more filters whose wavelength dependence of light transmittance differs from each other,
the light transmittance of each of the two or more filters has a maximum value in two or more wavelength ranges included in the more than three wavelength ranges.

### Aspect 5

The examining system according to any one of aspects 1 to 4, wherein
the imaging unit includes an imaging device having the image sensor,
the examining system further comprises:
a driving unit for moving the imaging device; and
a control circuit for controlling the driving unit,
the control circuit calculates an amount of displacement between a position of the fundus in an image indicated by the image information and a predetermined position, and controls the driving unit to move the imaging device based on the amount of displacement so as to reduce the amount of displacement.

### Aspect 6

The examining system according to any one of aspects 1 to 5, further comprising:
a light-shielding portion for shielding an area around at least one eye of the subject.

### Aspect 7

The examining system according to aspect 6, wherein
the light-shielding portion includes:
a front wall portion facing a front of a head of the subject; and
a support portion that supports the front wall portion,
the imaging unit is supported by the front wall portion, and wherein
the support portion maintains a predetermined distance between at least one eye of the subject and the imaging unit.

### Aspect 8

The examining system according to any one of aspects 1 to 7, further comprising:
a light source that emits light to irradiate at least one eye of the subject; and
a diffuser plate that diffuses light emitted from the light source.

### Aspect 9

The examining system according to any one of aspects 1 to 8, further comprising:
a guide light source that emits guide light to direct a line of sight of the subject toward the imaging unit.

### Aspect 10

The examining system according to any one of aspects 1 to 9, further comprising:
a wearable device attached to a head of the subject, wherein
the wearable device has the imaging unit.

### Aspect 11

The examining system according to any one of aspects 1 to 10, wherein
light incident on the imaging unit includes infrared light.

### Aspect 12

The examining system according to any one of aspects 1 to 11, wherein
the biometric information includes a blood glucose level.

### Aspect 13

An image processing system for examining biometric information of a subject, comprising:
an interface for acquiring image information used to determine the biometric information of subject from an imaging unit, the imaging unit simultaneously capturing light components in more than three wavelength ranges contained in light reflected from a fundus of the subject to generate the image information;
an image processing circuit that generates an image of the fundus for each wavelength range from the image information; and
a determination processing circuit that determines the biometric information of the subject based on the image of the fundus for each wavelength range.

### Aspect 14

A non-transitory computer-readable storage medium storing an examining program for examining biometric information of a subject, the program causing a computer to execute processes comprising:
acquiring image information used to determine the biometric information of the subject from an imaging unit, the imaging unit simultaneously capturing light components in more than three wavelength ranges contained in light reflected from a fundus of the subject to generate the image information;
generating an image of the fundus for each wavelength range from the image information; and
determining the biometric information of the subject based on the image of the fundus for each wavelength range.

### Aspect 15

A wearable device attached to a head of a subject, comprising:
an imaging unit including an image sensor, the imaging unit simultaneously capturing, by the image sensor, light components in more than three wavelength ranges contained in light reflected from a fundus of the subject to generate image information used to determine the biometric information of the subject; and
a light-shielding portion for shielding an area around at least one eye of the subject.

### Aspect 16

An examining method for examining biometric information of a subject, comprising:
imaging a fundus of the subject using an image sensor to generate image information used to determine the biometric information of the subject, the image sensor simultaneously capturing light components in more than three wavelength ranges contained in incident light;
generating, by an image processing circuit, an image of the fundus for each wavelength range from the image information; and
determining, by a determination processing circuit, the biometric information of the subject based on the image of the fundus for each wavelength range.

The present disclosure is applicable to determining subject's biometric information using a fundus image.

What is claimed is:

1. An examining system for examining biometric information of a subject, comprising:
an imaging unit including an image sensor, the imaging unit simultaneously capturing, by the image sensor, light components in more than three wavelength ranges contained in light reflected from a fundus of the subject to generate image information used to determine the biometric information of the subject;
an image processing circuit that generates an image of the fundus for each wavelength range from the image information; and
a determination processing circuit that determines the biometric information of the subject based on the image of the fundus for each wavelength range.

2. The examining system according to claim 1, wherein
the determination processing circuit acquires a light intensity spectrum of light reflected from retinal blood vessels contained in the fundus based on the image of the fundus for each wavelength range, and determines the biometric information of the subject based on the light intensity spectrum acquired.

3. The examining system according to claim 1, wherein
the image sensor has a plurality of pixels, outputs a signal in which pieces of information from each wavelength range are superimposed from each of the plurality of pixels, and generates the image information using the signal output from each of the plurality of pixels.

4. The examining system according to claim 3, wherein
the imaging unit further includes a filter array positioned in an optical path of light incident on the image sensor,
the filter array has a plurality of light-transmitting filters arranged two-dimensionally along a plane intersecting the optical path,
the plurality of filters includes two or more filters whose wavelength dependence of light transmittance differs from each other,
the light transmittance of each of the two or more filters has a maximum value in two or more wavelength ranges included in the more than three wavelength ranges.

5. The examining system according to claim 1, wherein
the imaging unit includes an imaging device having the image sensor,
the examining system further comprises:
a driving unit for moving the imaging device; and
a control circuit for controlling the driving unit,
the control circuit calculates an amount of displacement between a position of the fundus in an image indicated by the image information and a predetermined position, and controls the driving unit to move the imaging device based on the amount of displacement so as to reduce the amount of displacement.

6. The examining system according to claim 1, further comprising:
a light-shielding portion for shielding an area around at least one eye of the subject.

7. The examining system according to claim 6, wherein
the light-shielding portion includes:
a front wall portion facing a front of a head of the subject; and
a support portion that supports the front wall portion,
the imaging unit is supported by the front wall portion,
the support portion maintains a predetermined distance between at least one eye of the subject and the imaging unit.

8. The examining system according to claim 1, further comprising:
a light source that emits light to irradiate at least one eye of the subject; and
a diffuser plate that diffuses light emitted from the light source.

9. The examining system according to claim 1, further comprising:
a guide light source that emits guide light to direct a line of sight of the subject toward the imaging unit.

10. The examining system according to claim 1, further comprising:
a wearable device attached to a head of the subject, wherein
the wearable device has the imaging unit.

11. The examining system according to claim 1, wherein
light incident on the imaging unit includes infrared light.

12. The examining system according to claim 1, wherein
the biometric information includes a blood glucose level.

13. An image processing system for examining biometric information of a subject, comprising:
an interface for acquiring image information used to determine the biometric information of the subject from an imaging unit, the imaging unit simultaneously capturing light components in more than three wavelength ranges contained in light reflected from a fundus of the subject to generate the image information;
an image processing circuit that generates an image of the fundus for each wavelength range from the image information; and
a determination processing circuit that determines the biometric information of the subject based on the image of the fundus for each wavelength range.

14. A non-transitory computer-readable storage medium storing an examining program for examining biometric information of a subject, the program causing a computer to execute processes comprising:
acquiring image information used to determine the biometric information of the subject from an imaging unit, the imaging unit simultaneously capturing light components in more than three wavelength ranges contained in light reflected from a fundus of the subject to generate the image information;
generating an image of the fundus for each wavelength range from the image information; and
determining the biometric information of the subject based on the image of the fundus for each wavelength range.

15. A wearable device attached to a head of a subject, comprising:
an imaging unit including an image sensor, the imaging unit simultaneously capturing, by the image sensor, light components in more than three wavelength ranges contained in light reflected from a fundus of the subject to generate image information used to determine the biometric information of the subject; and
a light-shielding portion for shielding an area around at least one eye of the subject.

16. An examining method for examining biometric information of a subject, comprising:
imaging a fundus of the subject using an image sensor to generate image information used to determine the biometric information of the subject, the image sensor simultaneously capturing light components in more than three wavelength ranges contained in incident light;
generating, by an image processing circuit, an image of the fundus for each wavelength range from the image information; and
determining, by a determination processing circuit, the biometric information of the subject based on the image of the fundus for each wavelength range.
